(19) United States
(12) Patent Application Publication
(10) Pub. No.: US 2024/0238604 A1
(43) Pub. Date: Jul. 18, 2024

(54) ELECTRODE ATTACHMENT IN WEARABLE CARDIAC DEVICES

(71) Applicant: ZOLL Medical Corporation, Chelmsford, MA (US)

(72) Inventors: Saundra L. Penkunas, Portland, OR (US); Krista Marie Bacchioni, New Albany, OH (US); Stephanie Kristin Muhlenfeld, Portland, OR (US)

(21) Appl. No.: 18/413,819

(22) Filed: Jan. 16, 2024

Related U.S. Application Data
(60) Provisional application No. 63/480,387, filed on Jan. 18, 2023.

(51) Int. Cl. A61N 1/39; A61B 5/00; A61B 5/282; A61B 5/33; A61B 5/364

(52) U.S. Cl. CPC A61N 1/3904; A61B 5/282; A61B 5/33; A61B 5/364; A61B 5/6805; A61B 2562/222

(57) ABSTRACT

Ambulatory cardiac devices for providing comfortable, long-term continuous cardiac monitoring and treatment for arrythmia conditions. Examples of a device include sensing electrodes configured to detect ECG signals of an ambulatory patient, a garment configured to be worn about the patient's thorax, and sensing electrode receptacles configured to dispose, via the garment, the sensing electrodes at predetermined anatomical locations on the patient's thorax, and maintain, via the garment, contact between the sensing electrodes and the predetermined anatomical locations despite movement of the patient's thorax. In examples, each sensing electrode receptacle forms an opening in the garment and includes a securement device configured to allow for removable installation of a sensing electrode, a lock configured to inhibit movement of the sensing electrode separate from the sensing electrode receptacle, and a guide configured to align the sensing electrode with one of the predetermined anatomical locations of the patient's thorax through the opening.

### CROSS-REFERENCE TO RELATED APPLICATIONS

This application claims priority under 35 U.S.C. § 119(e) to U.S. Provisional Application No. 63/480,387 filed on Jan. 18, 2023 and titled “ELECTRODE ATTACHMENT IN WEARABLE CARDIAC DEVICES,” which is hereby incorporated herein by reference in its entirety for all purposes.

### TECHNICAL FIELD

The present disclosure is directed to an ambulatory cardiac systems, devices, and techniques for providing comfortable, long-term continuous cardiac monitoring and treatment for cardiac conditions.

### BACKGROUND

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. As the wearable device is generally prescribed for continuous use (e.g., only to be removed when bathing), the patient wears the device during all daily activities such as walking, sitting, climbing stairs, resting or sleeping, and other similar daily activities. Maintaining continuous use of the device as prescribed can be beneficial for monitoring patient progress as well as providing treatment to the patient if needed.

### SUMMARY

According to at least one embodiment, there is provided an ambulatory cardiac device for providing comfortable, long-term continuous cardiac monitoring and treatment for arrythmia conditions. Embodiments of the device comprise a plurality of sensing electrodes configured to detect electrocardiogram (ECG) signals of an ambulatory patient, a garment configured to be worn about the patient's thorax, and a plurality of sensing electrode receptacles configured to dispose, via the garment, the plurality of sensing electrodes at a plurality of predetermined anatomical locations on the patient's thorax, and maintain, via the garment, contact between the plurality of sensing electrodes and the plurality of predetermined anatomical locations despite movement of the patient's thorax, wherein each sensing electrode receptacle forms an opening in the garment and comprises a securement device configured to allow for removable installation of (e.g., installation into, and removal from, the sensing electrode receptacle) a respective sensing electrode, a lock configured to inhibit movement of the respective sensing electrode separate from the sensing electrode receptacle, and a guide configured to align the respective sensing electrode with one of the plurality of predetermined anatomical locations of the patient's thorax through the opening. Embodiments of the device may further comprise a plurality of removable therapy electrodes configured to deliver a treatment to the patient in response to the ambulatory cardiac device detecting a cardiac arrhythmia condition indicated by the ECG signals.

Examples of the device may include any one or more of the following features.

In one example, the plurality of sensing electrodes is a plurality of first sensing electrodes, and the device further comprises at least one second sensing electrode permanently integrated with the garment.

In one example, the at least one second sensing electrode is stitched into the garment.

In another example, the at least one second sensing electrode is woven into the garment.

In one example, the securement device includes a first annular holder at least partially surrounding the opening. The lock may include a plurality of grooves formed in a surface of the annular holder, wherein at least one groove of the plurality of grooves engages the respective sensing electrode to lock the respective sensing electrode into the securement device. The guide may include a second annular holder coupled to the first annular holder and at least partially surrounding the opening. In one example, the first and second annular holders are made of a thermoplastic material.

In one example, each sensing electrode receptacle further includes an attachment device coupled to at least one of the guide and the securement device, the attachment device configured to secure the sensing electrode receptacle to the garment. The attachment device may be made of fabric, for example.

In one example, the plurality of sensing electrodes and the plurality of sensing electrode receptacles are configured as color-coded pairs, each color-coded pair including one sensing electrode and one sensing electrode receptacle.

In one example, the garment comprises a body region, a pair of side portions extending laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions.

The garment may further comprise a belt flap configured to be removably attached to at least one of the lower portion of the body region or the pair of side portions. In one example, when attached to the at least one of the lower portion of the body region or the pair of side portions, the belt flap is disposed over the plurality of sensing electrode receptacles.

In another example, the garment further comprises a covering component configured to be removably attached to the upper portion of the body region and at least one of the lower portion of the body region or the pair of side portions. In one example, when attached to the upper portion of the body region and at least one of the lower portion of the body region or the pair of side portions, the covering component is disposed over the plurality of sensing electrode receptacles.

In one example, the device may further comprise a cabling harness coupled to the plurality of sensing electrodes, the cabling harness including at least one wire electrically connected to each respective sensing electrode. In one example, each sensing electrode receptacle includes a cable guide configured to secure the at least one wire electrically connected to the respective sensing electrode.

In one example, the garment comprises a plurality of pockets configured to removably house the plurality of therapy electrodes.

In another example, the securement device comprises a pouch secured to the garment, the pouch having a first opening to receive the respective sensing electrode. In one example, the guide comprises a second opening in the pouch, the second opening being aligned with the opening in the garment and configured to permit the respective sensing electrode to contact the patient's skin at the one of the predetermined anatomical locations of the patient's thorax through the second opening. The pouch may be made of a flexible polymer, for example. In one example, the securement device further comprises a fastener configured to at least partially close the first opening to secure the respective sensing electrode within the pouch.

Another embodiment provides a wearable cardiac device for providing comfortable, long-term continuous cardiac monitoring and treatment for arrythmia conditions. According to at least one embodiment, the device comprises a garment configured to be worn about a thorax of an ambulatory patient and having a plurality of garment openings formed therein, a plurality of sensing electrodes configured to detect electrocardiogram (ECG) signals of the patient, a plurality of sensing electrode receptacles secured to the garment and co-located with the plurality of garment openings, each sensing electrode receptacle of the plurality of sensing electrode receptacles being configured to removably secure a respective sensing electrode of the plurality of sensing electrodes at least partially within the sensing electrode receptacle with a portion of the respective sensing electrode extending through a respective garment opening of the plurality of garment openings, wherein each sensing electrode receptacle includes a lock configured to inhibit movement of the respective sensing electrode separate from the corresponding sensing electrode receptacle, and a plurality of therapy electrodes configured to deliver a treatment to the patient in response to the ambulatory cardiac device detecting a cardiac arrhythmia condition indicated by the ECG signals, the plurality of therapy electrodes being removably housed in the garment.

Examples of the device include any one or more of the following features.

In one example, the plurality of sensing electrodes is a plurality of first sensing electrodes, and the device further comprises at least one second sensing electrode permanently integrated with the garment. In one example, the at least one second sensing electrode is stitched into the garment. In another example, the at least one second sensing electrode is woven into the garment.

In one example, each sensing electrode receptacle comprises an annular holder at least partially surrounding the respective garment opening. In one example, the lock includes at least one ring formed in the annular holder and configured to engage the respective sensing electrode to lock the respective sensing electrode into the corresponding sensing electrode receptacle. In another example, the annular holder includes a first semi-rigid annular portion disposed on a first side of the garment and a second semi-rigid annular portion disposed on a second side of the garment and coupled to the first semi-rigid annular portion. Each sensing electrode receptacle may include at least one fastener configured to attach the first semi-rigid annular portion to the second semi-rigid annular portion.

In one example, each sensing electrode receptacle further comprises an attachment device configured to secure the sensing electrode receptacle to the garment. The attachment device may include an annular fabric component, for example.

In another example, each sensing electrode receptacle comprises a pouch having a first opening to receive the respective sensing electrode and a second opening aligned with the respective garment opening, and wherein the pouch is configured to removably secure the respective sensing electrode with the portion of the respective sensing electrode extending through the second opening and through the respective garment opening. In one example, the pouch has a semi-circular profile. The pouch may be made of a flexible polymer, such as a thermoplastic material, for example. In one example, the pouch is made of silicone.

In one example, the lock includes at least one protrusion formed on an interior of the pouch at an end of the pouch opposing the first opening, the at least one protrusion configured to engage the respective sensing electrode to secure the respective sensing electrode within the pouch.

In another example, each sensing electrode receptacle further comprises a securement component configured to at least partially close the first opening to secure the respective sensing electrode within the pouch. In one example, the securement component includes a hook and loop fastener. In another example, the securement component includes a snap fastener.

In one example, the plurality of sensing electrodes and the plurality of sensing electrode receptacles are configured as color-coded pairs, each color-coded pair including one sensing electrode and one sensing electrode receptacle.

In another example, each sensing electrode receptacle has a color different from the color of the other sensing electrode receptacles of the plurality of sensing electrode receptacles, and wherein the respective sensing electrode has a color indicator matching the color of the corresponding sensing electrode receptacle.

In one example, the garment comprises a body region, a pair of side portions extending generally laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions. The plurality of garment openings may be formed in at least one of the lower portion of the body region or the pair of side portions, for example.

In one example, the garment further comprises a belt flap configured to be removably attached to at least one of the lower portion of the body region or the pair of side portions. In one example, when attached to the at least one of the lower portion of the body region or the pair of side portions, the belt flap is disposed over the plurality of sensing electrode receptacles.

In another example, the garment further comprises a covering component configured to be removably attached to the upper portion of the body region and at least one of the lower portion of the body region or the pair of side portions. In one example, when attached to the upper portion of the body region and at least one of the lower portion of the body region or the pair of side portions, the covering component is disposed over the plurality of sensing electrode receptacles.

The device may further comprise a cabling harness coupled to the plurality of sensing electrodes, the cabling harness including at least one wire electrically connected to each respective sensing electrode. In one example, each sensing electrode receptacle includes a cable guide configured to secure the at least one wire electrically connected to the respective sensing electrode.

In one example, the garment comprises a plurality of pockets configured to removably house the plurality of therapy electrodes.

In one example, the device further comprises a controller electrically coupled to the plurality of therapy electrodes and to the plurality of sensing electrodes.

Another embodiment is directed to an easy-to-assemble, wearable cardiac device for providing comfortable, long-term continuous cardiac monitoring and treatment for arrythmia conditions. In at least one embodiment, the device comprises a garment configured to be worn about a thorax of a patient and having a plurality of garment openings formed therein, a plurality of sensing electrodes configured to detect electrocardiogram (ECG) signals of the patient, and a plurality of sensing electrode receptacles secured to the garment and co-located with the plurality of garment openings. The plurality of sensing electrode receptacles are configured to dispose, via the garment, the plurality of sensing electrodes at predetermined anatomical locations of the patient's thorax, and maintain, via the garment, contact between the plurality of sensing electrodes and the predetermined anatomical locations despite movement of the patient's thorax. Each sensing electrode receptacle comprises a pouch secured to the garment, the pouch having a first opening to receive a respective sensing electrode of the plurality of sensing electrodes and a second opening aligned with a respective garment opening of the plurality of garment openings and configured to permit the respective sensing electrode to contact the patient's skin at one of the predetermined anatomical locations of the patient's thorax through the second opening and the respective garment opening. The device may further comprise a plurality of removable therapy electrodes configured to deliver a treatment to the patient in response to the wearable cardiac device detecting a cardiac arrhythmia condition indicated by the ECG signals, the plurality of therapy electrodes being removably housed in the garment.

Examples of the device may include any one or more of the following features.

In one example, the plurality of sensing electrodes is a plurality of first sensing electrodes, and the device further comprises at least one second sensing electrode permanently integrated with the garment. The at least one second sensing electrode may be stitched or woven into the garment, for example.

In one example, each sensing electrode receptacle further comprises a securement component configured to removably secure the respective sensing electrode within the pouch. In one example, the securement device includes a fastener configured to at least partially close the first opening to secure the respective sensing electrode within the pouch. The fastener may be a hook and loop fastener or a snap fastener, for example. In another example, the securement component further includes at least one protrusion formed on an interior of the pouch and configured to engage the respective sensing electrode to secure the respective sensing electrode within the pouch.

In one example, the pouch is made of a flexible polymer. In another example, the pouch is made of silicone.

In one example, the device may further comprise a cabling harness coupled to the plurality of sensing electrodes, the cabling harness including at least one wire electrically connected to each respective sensing electrode. In one example, each sensing electrode receptacle further comprises a cable guide configured to secure the at least one wire electrically connected to the respective sensing electrode. The cable guide may include a pair of slits formed in the pouch, for example.

In another example, the plurality of sensing electrodes and the plurality of sensing electrode receptacles are configured as color-coded pairs, each color-coded pair including one sensing electrode and one sensing electrode receptacle.

In another example, the garment comprises a body region, a pair of side portions extending laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions. In one example, the garment further comprises a belt flap configured to be removably attached to at least one of the lower portion of the body region or the pair of side portions. In one example, when attached to the at least one of the lower portion of the body region or the pair of side portions, the belt flap is disposed over the plurality of sensing electrode receptacles. In another example, the garment further comprises a covering component configured to be removably attached to the upper portion of the body region and at least one of the lower portion of the body region or the pair of side portions. In one example, when attached to the upper portion of the body region and at least one of the lower portion of the body region or the pair of side portions, the covering component is disposed over the plurality of sensing electrode receptacles.

In one example, the garment comprises a plurality of pockets configured to removably house the plurality of therapy electrodes.

According to another embodiment, a wearable cardiac device for providing comfortable, long-term continuous cardiac monitoring and treatment for arrythmia conditions comprises a garment configured to be worn about a thorax of an ambulatory patient, a plurality of sensing electrodes configured to detect electrocardiogram (ECG) signals of the patient, and a plurality of sensing electrode receptacles secured to the garment and configured to dispose, via the garment, the plurality of sensing electrodes at predetermined anatomical locations of the patient's thorax, and maintain, via the garment, contact between the plurality of sensing electrodes and the predetermined anatomical locations despite movement of the patient's thorax. Each sensing electrode receptacle may comprise a retaining portion secured to the garment, and a movable portion coupled to the retaining portion and movable between a first position and a second position, a respective sensing electrode of the plurality of sensing electrodes being coupled to the movable portion, and wherein each electrode receptacle is configured to hold the respective sensing electrode in contact with the thorax of the patient when the movable portion is in the second position to permit detection of the ECG signals. The device further comprises a plurality of removable therapy electrodes configured to deliver a treatment to the patient in response to the wearable cardiac device detecting a cardiac arrhythmia condition indicated by the ECG signals, the plurality of therapy electrodes being removably housed in the garment.

Examples of the device may include any one or more of the following features.

In one example, the plurality of sensing electrodes is a plurality of first sensing electrodes, and the device further comprises at least one second sensing electrode permanently integrated with the garment. The at least one second sensing electrode may be stitched or woven into the garment, for example.

In one example, the retaining portion forms an opening, and wherein the movable portion is configured, when in the second position, to extend the respective sensing electrode through the opening. In another example, the garment comprises a plurality of garment openings and the plurality of sensing electrode receptacles are co-located with the plurality of garment openings such that the opening in the retaining portion is aligned with a respective garment opening of the plurality of garment openings.

In one example, the movable portion is integrally formed with the retaining portion. The retaining portion and the movable portion may be made of silicone, for example.

In one example, each sensing electrode receptacle further comprises a securement device configured to removably secure the respective sensing electrode to the movable portion of the electrode receptacle. In one example, the securement device includes a ring formed on the movable portion and configured to engage the respective sensing electrode. In another example, the securement device includes an adhesive.

In one example, the device further comprises a cabling harness coupled to the plurality of sensing electrodes, the cabling harness including at least one wire electrically connected to each respective sensing electrode. Each sensing electrode receptacle may further comprise a cable guide configured to secure the at least one wire electrically connected to the respective sensing electrode.

In another example, the plurality of sensing electrodes and the plurality of sensing electrode receptacles are configured as color-coded pairs, each color-coded pair including one sensing electrode and one sensing electrode receptacle.

In another example, the garment comprises a body region, a pair of side portions extending laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions. The garment may further comprise a belt flap configured to be removably attached to at least one of the lower portion of the body region or the pair of side portions. In one example, when attached to the at least one of the lower portion of the body region or the pair of side portions, the belt flap is disposed over the plurality of sensing electrode receptacles. In another example, the garment further comprises a covering component configured to be removably attached to the upper portion of the body region and at least one of the lower portion of the body region or the pair of side portions. In one example, when attached to the upper portion of the body region and at least one of the lower portion of the body region or the pair of side portions, the covering component is disposed over the plurality of sensing electrode receptacles.

The garment may further comprise a plurality of pockets configured to removably house the plurality of therapy electrodes.

According to another embodiment, a wearable cardiac device for providing comfortable, long-term continuous cardiac monitoring and treatment for arrythmia conditions comprise a garment configured to be worn about a thorax of an ambulatory patient, a plurality of sensing electrodes configured to detect electrocardiogram (ECG) signals of the patient, and an electrode attachment device configured to removably secure the plurality of sensing electrodes to the garment. The electrode attachment device comprises a plurality of first hook and loop fastener components, each first hook and loop fastener component of the plurality of first hook and loop fastener components being attached to a respective sensing electrode of the plurality of sensing electrodes, a corresponding plurality of second hook and loop fastener components attached to the garment and configured to engage with the plurality of first hook and loop fastener components to removably secure the sensing electrodes to the garment, and at least one securement component configured to align the sensing electrodes with respect to the plurality of second hook and loop fastener components. The wearable cardiac device may further comprise a plurality of removable therapy electrodes configured to deliver a treatment to the patient in response to the wearable cardiac device detecting a cardiac arrhythmia condition indicated by the ECG signals, the plurality of therapy electrodes being removably housed in the garment.

Examples of the wearable cardiac device may include any one or more of the following features.

In one example, each sensing electrode of the plurality of sensing electrodes has a first side and an opposing second side, and wherein each first hook and loop fastener is attached to the first side of the respective sensing electrode.

In one example, the at least one securement component comprises a plurality of semi-rigid securement components, each semi-rigid securement component being positioned around a respective second hook and loop fastener component of the plurality of second hook and loop fastener components and configured to engage a corresponding respective sensing electrode of the plurality of sensing electrodes to align the corresponding respective sensing electrode with the respective second hook and loop fastener component. Each semi-rigid securement component may be made of a thermoplastic or thermopolymer material, for example.

In one example, the plurality of sensing electrodes is a plurality of first sensing electrodes, and the device further comprises at least one second sensing electrode permanently integrated with the garment. The at least one second sensing electrode may be stitched or woven into the garment, for example.

In one example, the wearable cardiac device further comprises a cabling harness coupled to the plurality of sensing electrodes, the cabling harness including at least one wire electrically connected to each respective sensing electrode. In one example, each sensing electrode receptacle further comprises a cable guide configured to secure the at least one wire electrically connected to the respective sensing electrode.

In another example, the plurality of sensing electrodes and the plurality of electrode attachment devices are configured as color-coded pairs, each color-coded pair including one sensing electrode and one sensing electrode receptacle.

In another example, the garment comprises a body region, a pair of side portions extending laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions.

In one example, the garment further comprises a belt flap configured to be removably attached to at least one of the lower portion of the body region or the pair of side portions. When attached to the at least one of the lower portion of the body region or the pair of side portions, the belt flap may be disposed over the plurality of sensing electrode receptacles. In one example, the at least one securement component is disposed on the belt flap.

In another example, the garment further comprises a covering component configured to be removably attached to the upper portion of the body region and at least one of the lower portion of the body region or the pair of side portions. When attached to the upper portion of the body region and at least one of the lower portion of the body region or the pair of side portions, the covering component may be disposed over the plurality of sensing electrode receptacles. In one example, the at least one securement component is disposed on the covering component.

In another example, the garment comprises a plurality of pockets configured to removably house the plurality of therapy electrodes.

Another embodiment is directed to a support garment for housing a patient-worn cardiac monitoring apparatus and providing a comfortable fit to a thorax of a patient during long term continuous cardiac monitoring of the patient. At least one embodiment of the support garment comprises a body region, a pair of side portions extending generally laterally from either side of a lower portion of the body region, the side portions being attachable to each other to form a waist for the support garment, the lower portion and the pair of side portions including a plurality of openings formed therein, a pair of shoulder portions, each of said shoulder portions extending between an upper portion of the body region and a respective one of the side portions, and a plurality of sensing electrode receptacles, each sensing electrode receptacle being secured to one of the lower portion or one of the pair of side portions and at least partially surrounding a respective opening of the plurality of openings. Each sensing electrode receptacle may comprise an annular semi-rigid housing positioned around the respective opening and configured to removably secure a sensing electrode at least partially within the electrode receptacle, and a lock configured to inhibit movement of the respective sensing electrode separate from the sensing electrode receptacle.

Examples of the support garment may include any one or more of the following features.

In one example, the housing includes a first annular portion secured to a first side of the garment, and a second annular portion positioned on a second side of the garment and coupled to the first annular portion. In one example, the housing includes at least one fastener configured to attach the second annular portion to the first annular portion.

In another example, the lock includes a plurality of grooves formed in a surface of the housing and configured to engage the respective sensing electrode to lock the respective sensing electrode into the housing.

In one example, the housing is made of a thermoplastic material.

The support garment may further comprise at least one sensing electrode permanently integrated with the support garment. In one example, the at least one sensing electrode is stitched into the support garment. In another example, the at least one sensing electrode is woven into the support garment.

In one example, each sensing electrode receptacle further includes an attachment device coupled to the housing and configured to secure the sensing electrode receptacle to the support garment. The attachment device may be made of fabric, for example.

In another example, the support garment further comprises a belt flap configured to be removably attached to at least one of the lower portion of the body region or the pair of side portions. In one example, when attached to the at least one of the lower portion of the body region or the pair of side portions, the belt flap is disposed over the plurality of sensing electrode receptacles.

In another example, the support garment further comprises a covering component configured to be removably attached to the upper portion of the body region and at least one of the lower portion of the body region or the pair of side portions. In one example, when attached to the upper portion of the body region and at least one of the lower portion of the body region or the pair of side portions, the covering component is disposed over the plurality of sensing electrode receptacles.

According to another embodiment, an easy-to-assemble, wearable cardiac device for providing comfortable, long-term continuous cardiac monitoring and treatment for arrythmia conditions comprises a garment configured to be worn about a thorax of a patient and having a plurality of garment openings formed therein, a plurality of sensing electrodes configured to detect electrocardiogram (ECG) signals of the patient, and a plurality of sensing electrode receptacles secured to the garment and co-located with the plurality of garment openings. The plurality of sensing electrode receptacles may be configured to dispose, via the garment, the plurality of sensing electrodes at predetermined anatomical locations of the patient's thorax, and maintain, via the garment, contact between the plurality of sensing electrodes and the predetermined anatomical locations despite movement of the patient's thorax. In at least one example, each sensing electrode receptacle comprises a securement device configured to allow for removable installation of a respective sensing electrode at least partially within the sensing electrode receptable, and a retainer secured to the garment and having a first opening aligned with a respective garment opening of the plurality of garment openings and configured to permit the respective sensing electrode to contact the patient's skin at one of the predetermined anatomical locations of the patient's thorax through the first opening and the respective garment opening. At least one embodiment of the device further comprises a plurality of therapy electrodes configured to deliver a treatment to the patient in response to the wearable cardiac device detecting a cardiac arrhythmia condition indicated by the ECG signals, the plurality of therapy electrodes being removably housed in the garment.

Examples of the easy-to-assemble, wearable cardiac device may include any one or more of the following features.

In one example, the retainer comprises a pouch having the first opening and a second opening to receive a respective sensing electrode. In one example, the securement device comprises a fastener configured to at least partially close the first opening to secure the respective sensing electrode within the pouch.

In another example, each sensing electrode assembly further comprises a movable portion coupled to the retainer and movable between a first position and a second position, wherein the securement device is configured to removably secure the respective sensing electrode to the movable portion. The movable portion may be configured, when in the second position, to extend the respective sensing electrode through the first opening and the respective garment opening. Each electrode receptacle may be configured to hold the respective sensing electrode in contact with the thorax of the patient when the movable portion is in the second position to permit detection of the ECG signals. In one example, the movable portion is integrally formed with the retainer.

In another example, the garment comprises a body region, a pair of side portions extending laterally from either side of a lower portion of the body region, the side portions being attachable to each other to form a waist for the garment, and a pair of shoulder portions, each shoulder portion of the pair of shoulder portions extending between an upper portion of the body region and a respective one of the side portions.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

In the figures:

FIG. 1A is a diagram illustrating an example of an electrode belt that may be used with a wearable medical device;

FIG. 1B is a diagram illustrating an example of a wearable medical device;

FIG. 2 is a diagram illustrating an example of a support garment forming part of an ambulatory medical device in accordance with aspects of the present disclosure;

FIG. 3A is a diagram showing an example of ECG sensing electrode placement at various anatomical locations on a patient's body;

FIG. 3B is a diagram showing another example of ECG sensing electrode placement at various anatomical locations on a patient's body;

FIG. 4A is a diagram showing an example of ECG sensing electrode and therapy electrode placement at various anatomical locations on a patient's body;

FIG. 4B is a graph showing an example of ECG leads collected using an example of the sensing electrode placement arrangement shown in FIG. 4A;

FIG. 4C is a diagram illustrating pairing of sensing electrodes to produce ECG leads;

FIG. 4D is a diagram showing an example of planes through a patient's body corresponding to ECG lead projection;

FIG. 5A is a perspective view of one example of a sensing electrode receptacle in accordance with aspects of the present disclosure;

FIG. 5B is a side cross-sectional view of the sensing electrode receptacle of FIG. 5A;

FIG. 5C is a plan view of the sensing electrode receptacle of FIGS. 5A and 5B;

FIG. 6 is an exploded view of one example of an ECG sensing electrode assembly in accordance with aspects of the present disclosure;

FIG. 7A is a perspective view of an example of a portion of the sensing electrode receptacle of FIGS. 5A-C in accordance with aspects of the present disclosure;

FIG. 7B is a perspective view of one example of a base component for an example of an ECG sensing electrode assembly in accordance with aspects of the present disclosure;

FIG. 7C is a perspective view of an example of a portion of the sensing electrode receptacle of FIGS. 5A-C;

FIG. 7D is a plan view showing an example of the sensing electrode receptacle of FIGS. 5A-C including a locking mechanism to engage an ECG sensing electrode;

FIG. 7E is a plan view showing another example of the sensing electrode receptacle of

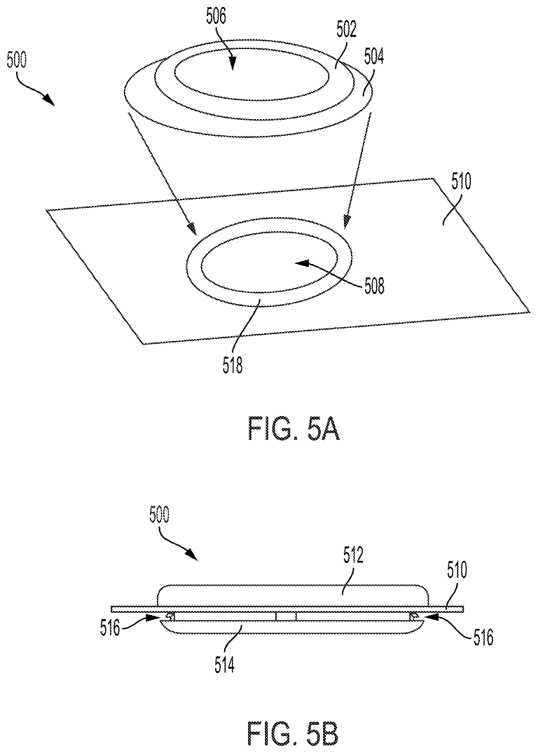

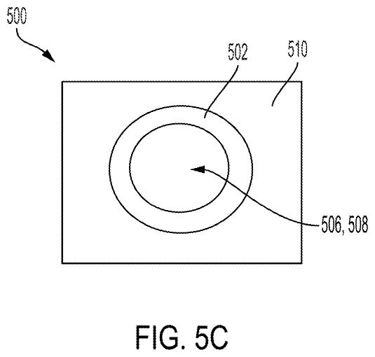

FIGS. 5A-C including a locking mechanism to engage an ECG sensing electrode;

FIG. 8A is a perspective view of another example of a sensing electrode receptacle in accordance with aspects of the present disclosure;

FIG. 8B is a perspective view illustrating an example of a sensing electrode installed within the sensing electrode receptacle of FIG. 8A;

FIG. 8C is a perspective view of another example of the sensing electrode receptacle of FIG. 8A in accordance with aspects of the present disclosure;

FIG. 8D is a perspective view of another example of a sensing electrode receptacle in accordance with aspects of the present disclosure;

FIG. 9 is a diagram illustrating an array of another example of sensing electrode receptacles in accordance with aspects of the present disclosure;

FIG. 10A is a diagram showing an example of a movable portion of a sensing electrode of

FIG. 9, the movable portion being in a first position;

FIG. 10B is a diagram showing an example of the movable portion of the sensing electrode of FIG. 9, the movable portion being in a second position;

FIG. 11A is a diagram showing an example of a support garment including a belt flap, the belt flap shown in an open position, in accordance with aspects of the present disclosure;

FIG. 11B is a diagram showing an example of the support garment including the belt flap, the belt flap shown in a closed position, in accordance with aspects of the present disclosure;

FIG. 12 is a diagram showing an example of a support garment including a cover, the cover shown in an open position, in accordance with aspects of the present disclosure;

FIG. 13A is a diagram showing another example of a support garment including a cover, the cover shown in an open position, in accordance with aspects of the present disclosure;

FIG. 13B is a diagram showing another example of a support garment including a cover, the cover shown in an open position, in accordance with aspects of the present disclosure;

FIG. 14 is a diagram showing another example of a support garment in accordance with aspects of the present disclosure;

FIG. 15 is a diagram showing an example of a sensing electrode assembly in accordance with aspects of the present disclosure;

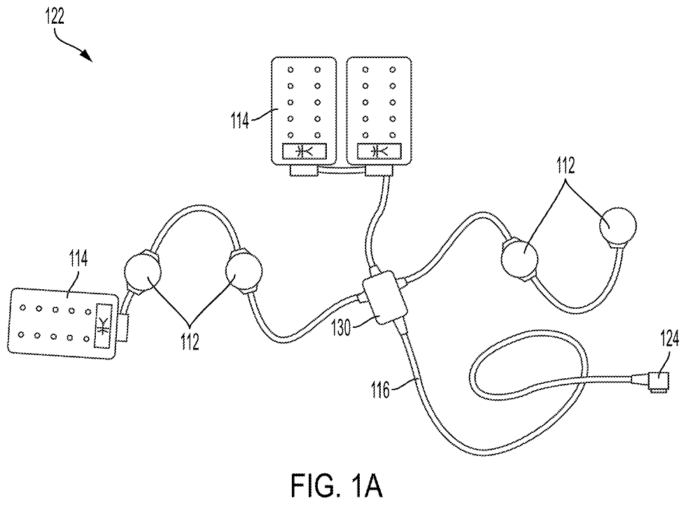

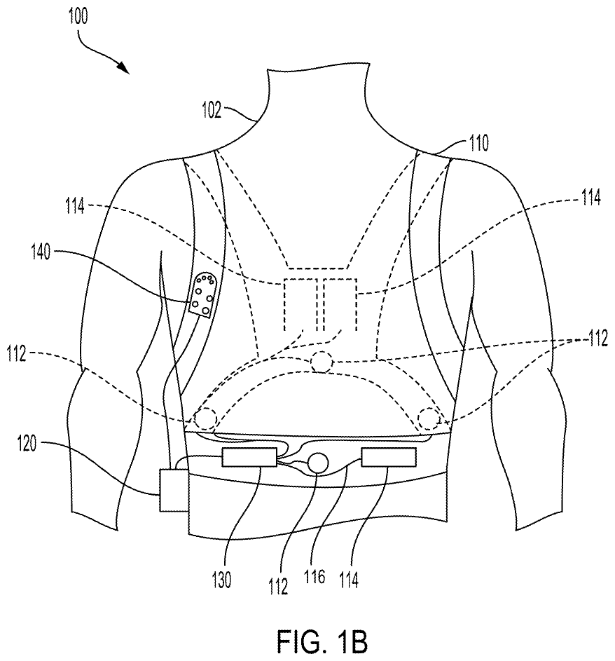

FIG. 16A is a block diagram illustrating a first view of a medical device controller for the wearable medical device of FIG. 1B;

FIG. 16B is a block diagram illustrating a second view of a medical device controller for the wearable medical device of FIG. 1B; and

FIG. 17 is a block diagram illustrating a schematic view of a sample controller for a wearable medical device, in accordance with aspects of the present disclosure.

### DETAILED DESCRIPTION

Cardiac monitoring and/or treatment systems include ECG sensing electrode systems that are used to measure electrical signals associated with the heart of a subject so that the systems can determine if the subject is exhibiting abnormal cardiac activity. A patient complaining of chest discomfort, pain, shortness of breath, or chest palpitations may be prescribed cardiac monitoring devices. A patient having an elevated risk of sudden cardiac death, unexplained syncope, prior symptoms of heart failure, an ejection fraction of, e.g., less than 45%, less than 35%, or other such predetermined threshold deemed of concern by a physician, and other similar patients in a state of degraded cardiac health can be prescribed specialized cardiac treatment devices. Examples of cardiac monitoring devices include a mobile cardiac telemetry (MCT) devices, continuous cardiac event monitoring (CEM) devices, and cardiac holter devices. Such cardiac monitoring devices are prescribed to study patients' cardiac activity over a time period to diagnose cardiac conditions and determine an appropriate therapy. Examples of cardiac arrhythmias assessed by cardiac monitoring devices and associated systems include bradycardias, tachyicardias, heart pauses, atrial fibrillation, ectopic beats, PVCs (including PVC runs and counts), biginimy, triginimy, n-giminy and the like, among other such cardiac conditions. Cardiac monitoring devices may be short term devices, e.g., intended for use over a 24 hour period, 48 hour period, 72 hour period, 1 week period, or 2 week period. Some cardiac monitoring devices may be prescribed for longer term, e.g., 30 days period, 60 days period, 90 days period, or 180 days period, or one (1) year period. These devices are configured to sense and record ECG signals, analyze such signals for cardiac arrhythmias, and/or transmit the ECG signals to a remote server for cardiac arrhythmia analysis, e.g., per-occurrence basis, or on a daily, weekly, monthly, or end of use basis. Cardiac treatment devices, on the other hand, are prescribed to treat life-threatening cardiac conditions such as ventricular tachycardia (VT) or ventricular fibrillation (VF). Such cardiac treatment devices include wearable cardioverter-defibrillators (WCDs), hospital wearable defibrillators (HWDs), and short term wearable defibrillators (SWDs). Short term cardiac treatment devices may be intended for use over a 24 hour period, 48 hour period, 72 hour period, 1 week period, or 2 week period. WCDs may be prescribed for longer term, e.g., 30 days period, 60 days period, 90 days period, or 180 days period, or one (1) year period. HWDs may be prescribe for a duration of hospital or other in-patient stay at a healthcare or cardiac rehabilitation facility.

For example, a wearable cardioverter defibrillator (WCD) in accordance with the systems, devices, and techniques as described herein can be worn by a patient at risk for sudden cardiac arrest and who is not a candidate for or refuses an implantable defibrillator. Such a device is configured to be lightweight and easy to assemble and wear as explained in further detail below, allowing patients to return to their activities of daily living. A WCD monitors the patient's heart continuously, and if the patient goes into a life-threatening, rapid heart rhythm, the WCD is configured to deliver a defibrillating or cardioverting treatment in an attempt to restore the patient's heart to normal rhythm. In such cases, the treatment is automatic and does not require bystanders to help.

Such medical devices can benefit from the incorporation of, or interoperation with, an adjustable garment configured to be worn about the torso of the patient. Further, given that wearable cardiac monitoring and/or treatment devices are prescribed for continuous and/or long term use, there is a need for the garment supporting the device to be comfortable and configured to reliably maintain skin contact between the ECG sensing electrodes and the patient's skin, even when the patient is moving (for example, during exercise or other movement). In examples, a WCD system includes an electrode belt and a garment configured to be worn about the patient's torso. The WCD system also includes a monitor that the patient carries around the waist or on a shoulder strap. Referring briefly to FIG. 1A, an example electrode belt 122 of a WCD is shown. The example electrode belt 122 includes the ECG sensing electrodes 112, therapy electrodes 114 (also referred to as therapy pads), a connection pod 130, a connector 124, and wiring or cables 116 configured to couple the ECG sensing electrodes 112, therapy pads 114, connection pod 130, and connector 124. The entire electrode belt 122, including the ECG sensing electrodes 112, is configured to be removably disposed in the garment as will be explained in more detail below.

In implementations as described herein, the electrode belt 122 including the ECG sensing electrodes 112 is configured to be easily removed from the garment so that the patient can wash the garment. In implementations as described herein, the electrode belt 122 can include one, two, three, or other predetermined number of ECG sensing electrodes 112 configured to be positioned in predetermined anatomical locations on the patient.

In examples, a WCD system can be configured to include a garment with one, two, three, or other predetermined number of ECG sensing electrodes configured to be permanently disposed within the garment, and other one, two, three or predetermined number of ECG sensing electrodes configured to be removably disposed within the garment, as discussed further below with reference to FIG. 1B. In such examples, the electrode belt 122 including the removable ECG sensing electrodes is configured to be easily removed from the garment so that the patient can wash the garment with remaining washable components such as the permanently disposed ECG sensing electrodes.

In scenarios as described above, the WCD garments are configured to be periodically washed to maintain hygiene and also restore elasticity to maintain the appropriate skin contact between the ECG sensing electrodes and the patient's skin. For example, the patient is instructed to remove the garment at least every 48 to 72 hours and switch to a different garment. The patient may be instructed to remove the electrode belt, monitor, or any other non-washable accessories connected to the garment prior to washing the garment. It is thus desirable that the process of removing and replacing the ECG sensing electrodes be easy for all types of patients relative to existing or conventional techniques. It is desirable to minimize the time needed to remove and replace the ECG sensing electrodes relative to existing or conventional techniques by making the assembly and disassembly of the electrode belt into and out of the garment as easy as possible.

The example ECG removal systems, devices, and/or techniques provided herein promote comfort, aesthetic appearance, and ease of use or application for older patients, or patients with physical infirmities and/or who are physically challenged, including patients with rheumatic conditions, patients with arthritis, and/or patients with autoimmune or inflammatory diseases that affect joints, tendons, ligaments, bones, and muscles of the arm and hand. Patients afflicted with such conditions can properly and/or correctly don the garments described herein as well as remove or attach the ECG sensing electrodes to such garments. The removable ECG sensing assemblies and associated features minimize the time needed by patients to assemble, don or remove the ECG sensing electrodes and/or the associated support garment. Further, patients benefit from such ECG sensing assemblies and associated features, which can facilitate longer wear times, better patient compliance, and improve the reliability of the detected physiological signals and treatment of the patient. These removable ECG sensing assemblies and associated features promote ease of use, comfort and an aesthetic appearance for such patient populations. For example, the removable ECG sensing assemblies and associated features as well as the garments described herein generally follow design principles as noted below (e.g., similar to those prescribed in the Arthritis Foundation Guidelines).
Removing, donning, and assembling the garment and associated components do not require fine motor control or simultaneous actions,
Replacing electrodes and other components is possible for patients with limited reach and strength,
The garment and/or components include surface and/or textural aspects that makes the garment and/or components easy to grip and control.
The garment and/or components include features designed to minimize simultaneous actions such as depressing and pulling,
The garment and/or components include features to provide positive feedback, including audible and/or tactile feedback (for example, “snap”, “click”, among others).

These features can encourage patients to wear the support garment and associated medical device for longer and/or continuous periods of time with minimal interruptions in the periods of wear. For example, by minimizing interruptions in periods of wear and/or promoting longer wear durations, patients and caregivers can be assured that the device is providing desirable information about as well as protection from adverse cardiac events such as ventricular tachycardia and/or ventricular fibrillation, among others. Moreover, when the patient's wear time and/or compliance is improved, the device can collect information on arrhythmias that are not immediately life-threatening, but may be useful to monitor for the patient's cardiac health.

As such, this disclosure relates to various devices and methods for attaching such ECG sensing electrode assemblies to a garment for wear by a patient. As discussed in more detail below, examples of the attachment mechanisms disclosed herein may allow for ease of assembly of the ECG sensing electrode assemblies with the garment, comfortable fit and feel for the patient, and secure, reliable positioning of the ECG sensing electrodes in appropriate locations on the patient's body for accurate cardiac monitoring.

In accord with the above, various embodiments are directed to wearable cardiac devices for providing comfortable, long-term continuous cardiac monitoring and treatment for arrythmia conditions. According to some examples, such a wearable cardiac device may include a plurality of sensing electrodes configured to detect ECG signals of an ambulatory patient, a garment configured to be worn about the patient's thorax, and a plurality of sensing electrode receptacles that are configured to dispose, via the garment, the plurality of sensing electrodes at a plurality of predetermined anatomical locations on the patient's thorax, and to maintain, via the garment, contact between the plurality of sensing electrodes and the plurality of predetermined anatomical locations despite movement of the patient's thorax. In some examples, the wearable cardiac device may also include a plurality of therapy electrodes that are removably housed in the garment and configured to deliver a treatment to the patient in response to the cardiac device detecting a cardiac arrhythmia condition indicated by the ECG signals.

According to some examples, each sensing electrode receptacle forms or is disposed about an opening in the garment to allow an installed sensing electrode to contact the patient's skin through the opening in the garment. A benefit of providing such an opening is to provide for a mechanical path for the installed sensing electrode of appropriate dimension and construction to traverse from one side of the garment (facing away from patient's skin) to the other side of the garment (facing towards the patient's skin) to thus contact the patient's skin. Alternatively or additionally, a benefit of providing such an opening is to provide for the construction and/or additional of other mechanical features noted below (e.g., securement device, lock, among others). The opening thus operates synergistically with other such mechanical features to provide an overall benefit of aesthetic appearance and ease of installation and removal of the corresponding ECG sensing electrode. In examples, a benefit of providing the opening is to provide for appropriate dimension and shape to accurately locate the sensing electrode at the predetermined anatomical location.

Examples of the sensing electrode receptacle include a securement device that is configured to allow for removable installation of the sensing electrode (e.g., to allow the sensing electrode to be installed into the sensing electrode receptacle and to be non-destructively removed from the sensing electrode receptacle). A benefit of providing such a securement device is to allow for the sensing electrode to be securely held in the sensing electrode receptacle, such that it does not fall out or move as the patient moves or as the patient handles the garment (e.g., when putting the garment on and/or taking it off). Examples of the securement device further offer benefits of allowing the patient to quickly, easily, and properly both secure the sensing electrode with the sensing electrode receptacle and remove the sensing electrode from the sensing electrode receptacle. The sensing electrode receptacle can include a casing that is secured to the garment. In some examples, the casing includes an annular holder or a pouch that can shaped and sized to receive and secure the sensing electrode. Providing such a casing offers a benefit in that it provides a first level of containment for the sensing electrode, holding it in roughly the correct location, while the patient can then engage the securement device, locking mechanism, and/or cable guides as discussed further below. As such, the casing can make it easier for the patient to correctly assemble the electrode belt 122 into the garment 110. Furthermore, in some examples the casing provides a benefit of being an easily visible part of the sensing electrode receptacle that can be color-coded and matched to color-coded sensing electrodes to make it easier for the patient to correctly match the sensing electrodes with the appropriate sensing electrode receptacles. The casing can also assist the patient in properly placing and aligning the sensing electrode. For example, as discussed further below, the casing can be sized and shaped in a manner that matches or corresponds to the sensing electrode, thus making it easier for the patient to correctly install the sensing electrode without allowing the sensing electrode to be misaligned or not properly secured in the sensing electrode assembly. In some examples, the securement device is implemented as part of the casing, as is discussed further below. In one example in which the casing includes a pouch, the securement device may include a fastener that at least partially closes the pouch after the sensing electrode has been installed. This offers the benefit of providing an easy-to-use feature that ensures that the sensing electrode is secured within the sensing electrode receptacle and cannot fall out as the patient moves or handles the garment. Various other examples of securement devices are discussed further below.

In some examples, the sensing electrode receptacle can include a lock configured to inhibit movement of the respective sensing electrode separate from the sensing electrode receptacle. Benefits of such locking features include minimizing the rotational and/or lateral shifting of the sensing electrode on the skin of the patient. Benefits of such locking features include providing tactile and visual feedback to the patient that the sensing electrode is properly secured within the electrode receptacle.

In some examples, the sensing electrode receptacle may include a guide configured to align the respective sensing electrode with one of the plurality of predetermined anatomical locations of the patient's thorax. In some instances, in order to achieve accurate ECG measurements, it is important that the sensing electrodes be correctly placed and aligned with the predetermined anatomical locations on the patient's thorax. Accordingly, a benefit to providing such a guide is to assist the patient in achieving the proper placement and alignment of the sensing electrodes quickly and easily. The guide may be implemented as part of the casing in some examples. In some examples, the guide may include an alignment component that is integrated with the garment and configured to engage the sensing electrode so as to align the sensing electrode with respect to the sensing electrode receptacle and/or a selected anatomical location on the patient's thorax. A benefit to providing the alignment component integrated with the garment is that the alignment component is thus correctly placed with respect to the predetermined anatomical locations on the patient's thorax without further effort (provided correct size and fit of the garment), and therefore can provide a reliable placement guide for the patient as the sensing electrodes are installed.

These examples, and various other similar examples that benefit from the techniques, processes, and approaches as provided herein, are described in additional detail below.

FIG. 1B illustrates an example medical device 100 that is external, ambulatory, and wearable by a patient 102, and configured to implement one or more configurations described herein. For example, the medical device 100 can be a non-invasive medical device configured to be located substantially external to the patient 102. Such a medical device 100 can be, for example, an ambulatory medical device that is capable of and designed for moving with the patient as the patient goes about his or her daily routine. Such wearable defibrillators typically are worn nearly continuously or substantially continuously for two to three months at a time. During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 100 can include one or more of the following: a garment 110, an ECG electrode assembly including one or more ECG sensing electrodes 112, one or more therapy electrodes 114, a medical device controller 120, a connection pod 130, a patient interface pod 140, or any combination of these. Examples of these components are discussed in more detail below. In some examples, at least some of the components of the medical device 100 can be configured to be affixed to the garment 110 (or in some examples, permanently integrated into the garment 110), which can be worn about the patient's torso.

Depending upon the manufacturing process and the intended wear instructions, the garment 110 can be manufactured from a variety of materials. For example, to provide a constant force against the physiological sensors such that the sensors maintain contact with a patient's body, the garment can be made from a material or a combination of materials that have elastic or other similar stretching characteristics. In some examples, the entire garment can be made from a material that is configured to be stretched and to return to its original shape. In other examples, the garment can be made from a combination of materials such that only a portion of the garment can be stretched and returned to its original shape. In certain implementations, the garment can be woven from one or more materials. Depending upon the type of material used and properties of the weave of the material, the elasticity of the garment can be controlled such that areas with a tighter fabric weave are less pliant than areas with a looser fabric weave. Examples of materials that can be used to manufacture a garment as described herein can include, for example, cotton, nylon, spandex, polyester, elastin, Lycra®, and other similar natural and synthetic materials. In some examples, materials can be combined to produce a garment such as a cotton/spandex combination or a nylon/spandex combination. It should be noted, however, that these materials are provided by way of example only and various other materials can be used to manufacture a garment as described herein.

The medical device controller 120 can be operatively coupled to the ECG sensing electrodes 112, which can be affixed to the garment 110, e.g., assembled into the garment 110 or removably attached to the garment 110, as discussed in more detail below. In some implementations, one or more of the sensing electrodes 112 can be permanently integrated into the garment 110. The medical device 100 may further include a cabling harness or assembly, including one or more cables/wires 116, that electrically connect various components of the medical device 100. For example, the cabling harness may electrically connect two or more of the sensing electrodes 112, and may connect the sensing electrodes 112 and/or therapy electrodes 114 to the medical device controller 120 and/or to the connection pod 130, as discussed further below. In some examples, the sensing electrodes 112 can be operatively coupled to the medical device controller 120 through the connection pod 130.

The sensing electrodes 112 can be configured to detect one or more cardiac signals. Examples of such signals include ECG signals and/or other sensed cardiac physiological signals from the patient. Accordingly, the sensing electrodes 112 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag-AgCl.

In some examples, the sensing electrodes 112 can be used with an electrolytic gel dispersed between the electrode surface and the patient's skin. In other examples, the ECG sensing electrodes 112 are implemented as “dry” ECG electrodes and as such do not include hydrogel or other conductive ECG gel disposed between the electrode surface and the patient's skin. According to certain examples, the sensing electrodes 112 are configured to be in contact with the patient's skin for continuous use and for extended periods of time, as discussed above. In examples where the ECG sensing electrodes are configured as dry electrodes, the ECG sensing electrodes are more comfortable against the patient's skin for continuous use scenarios and/or extended wear durations, including where such use is in the presence of high humidity and/or moisture. In addition, with the use of dry electrodes, the assembly, disassembly, and maintenance of the medical device 100 is convenient for the patient in such environs. Further, ECG sensing electrode assemblies as disclosed herein allow for easy donning and removal of the medical device 100. For example, in this regard, patients do not need to concern themselves with applying or re-applying conductive gel to the ECG sensing electrodes before, during, or after physical activities, shower, or bathing. Example dry sensing electrodes 112 include a metal electrode (e.g., a tantalum metal) with an oxide coating such as a tantalum pentoxide coating, for example.

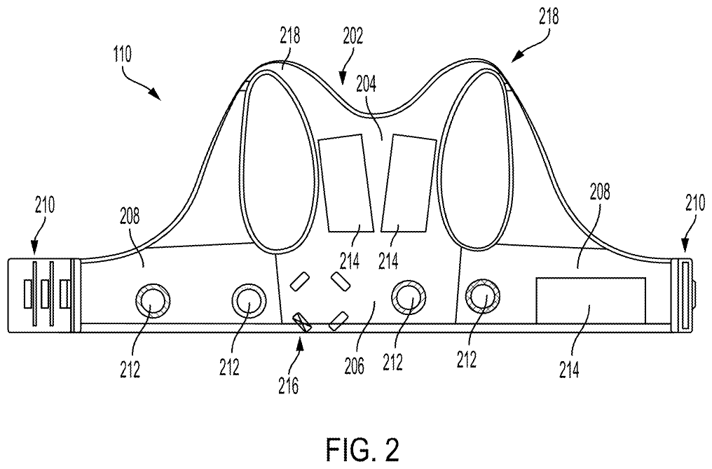

As discussed above, in examples of the medical device 100, the support garment 110 is worn by the patient and configured to support various components of the medical device 100, including the sensing electrodes 112 and therapy electrodes 114, for example. FIG. 2 is a schematic diagram illustrating an example of the support garment 110 according to certain aspects of this disclosure. The garment 110 may be made of a flexible fabric material designed for comfortable wear by the patient 102, as discussed above. The garment 110 includes a body region 202 having an upper portion 204 and a lower portion 206. The garment includes a pair of side portions 208 extending generally laterally from either side of the lower portion 206 of the body region 202. The side portions 208 are attachable to each other, e.g., using a fastening mechanism 210, to form a waist for the support garment 110. The fastening mechanism 210 may include a clip, one or more snap fasteners, hook and loop fasteners, one or more buttons, or another type of attachment mechanism. The garment further includes a pair of shoulder portions 218, each of the shoulder portions 218 extending between the upper portion 204 of the body region 202 and a respective one of the side portions 208, as shown in FIG. 2. When worn by the patient 102, the body region 202 and side portions 208 may be positioned around the patient's thorax, with the upper portion 204 of the body region 202 positioned against the patient's back, as shown in FIG. 1B, for example.

According to certain embodiments, and as discussed in more detail below, the support garment 110 may be configured to support an electrode system, such as the electrode belt 122 discussed above, that includes a plurality of sensing electrodes 112 along with a cabling harness (not shown in FIG. 2) coupled to the plurality of sensing electrodes 112, the cabling harness including at least one wire(s) or cable(s) 116 electrically connected to each respective sensing electrode. Accordingly, in some examples, the garment 110 may include a plurality of sensing electrode receptacles 212 that are configured to attach the sensing electrodes 112 to the garment 110. The sensing electrode receptacles 212 may be positioned on or in the lower portion 206 of the body region 202 and/or one or both of the side portions 208. Additional support/attachment features (not shown in FIG. 2) for supporting the cabling harness may be included in some examples, as discussed further below. In other examples, the cables or wires of the cabling harness may be sewn into the garment 110, or otherwise permanently integrated into the garment 110. In some examples, the garment 110 may further include a plurality of pockets 214 that house the therapy electrodes 114. In the illustrated example, the garment 110 includes two therapy electrode pockets 214 on the upper portion 204 of the body region 202 and one therapy electrode pocket 214 on one of the side portions 208; however, other arrangements are possible. The garment 110 may further include other features for attaching one or more other components of the medical device 100 to the garment, such as a fastening mechanism 216 for attaching the connection pod 130 and/or the medical device controller 120, for example.

In some examples, the garment 110 and the arrangement of the sensing electrode receptacles 212 can be configured to position the sensing electrodes 112 at particular anatomical locations on the patient's thorax when the patient is wearing the garment assembled with the sensing electrode system. The anatomical positions may be selected based on the ECG lead configuration used in the medical device 100.

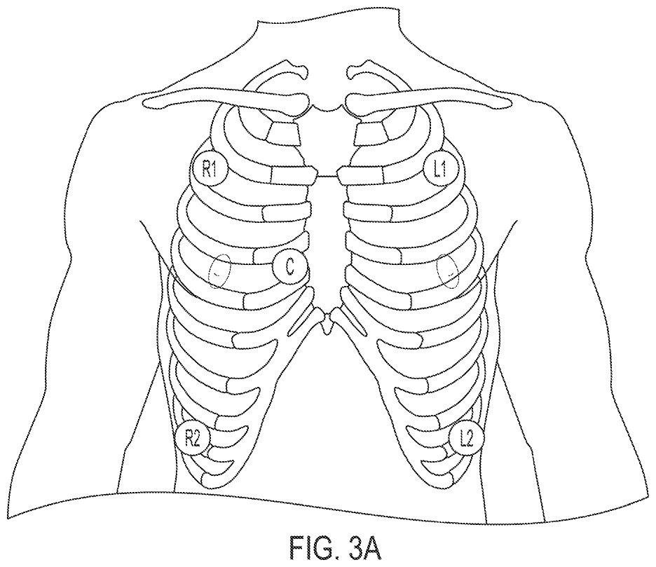

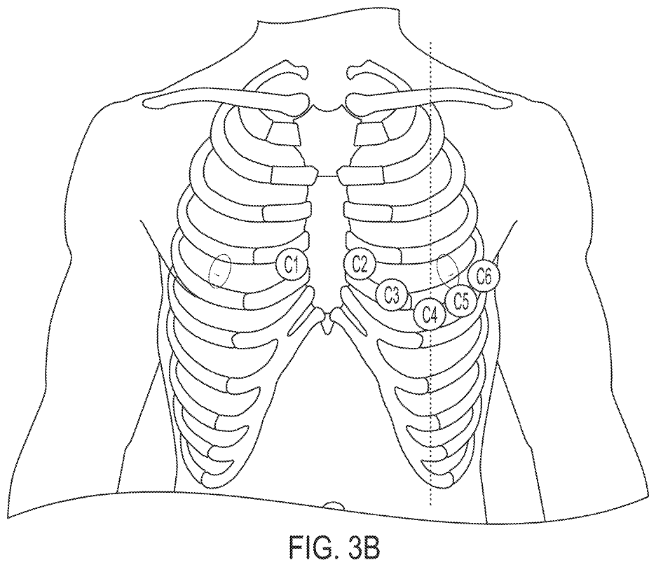

There are a variety of different ECG configurations that use different numbers of ECG sensing electrodes 112 and provide various ECG leads. Cardiac monitoring systems that produce ECG leads may be referred to as ECG lead systems. Referring to FIGS. 3A and 3B, some examples of ECG lead systems include three-lead systems, five-lead systems, and twelve-lead systems. For example, a three-lead system may use three sensing electrodes 112 that are located at predetermined anatomical locations including R1, L1, and L2, producing three ECG leads. For example, a five-lead system may use five sensing electrodes 112 that are located at predetermined anatomical locations including R1, R2, L1, L2, and a chest position, C, which in some examples may be a position selected from positions C1-6 (see FIG. 3B). In another example, a twelve-lead system uses ten sensing electrodes positioned on the patient's chest and limbs, and produces two sets of ECG leads, namely six limb leads and six chest leads. Chest leads may also be referred to as precordial leads.

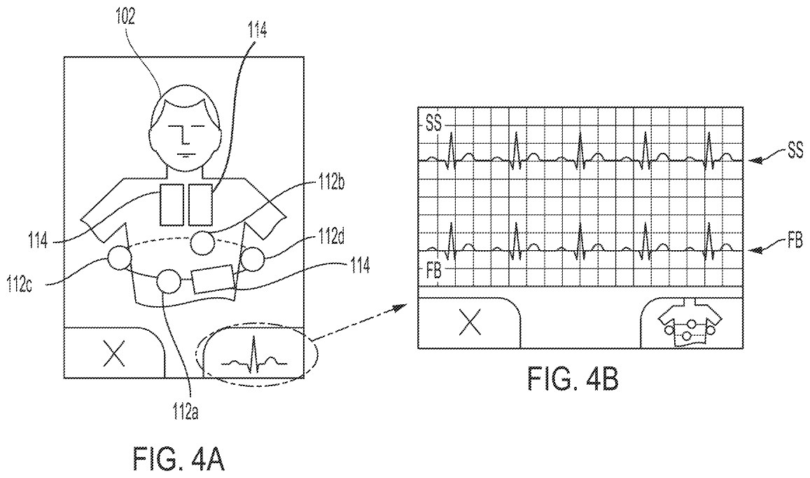

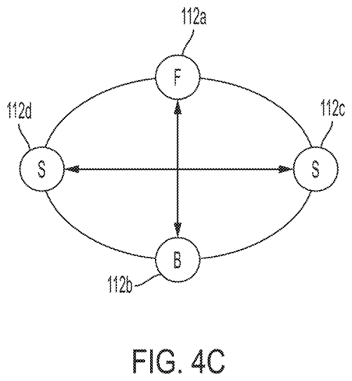

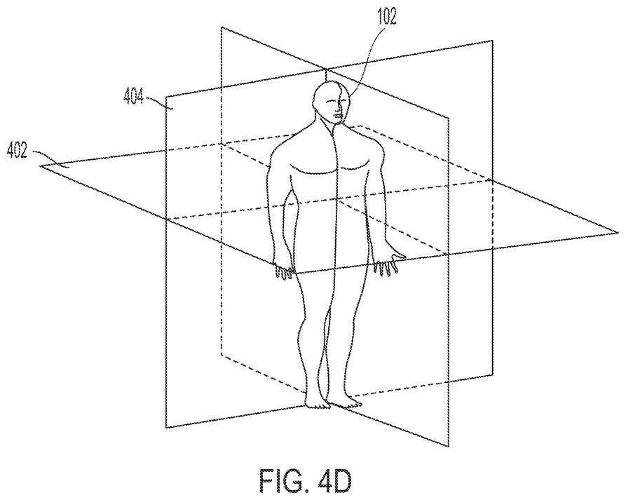

FIGS. 4A-D illustrates another example in which at least four ECG sensing electrodes 112 are disposed on the sides of the patient's body and on an anterior and posterior position of the patient's body, similar to the arrangement shown in FIG. 1B. In the example shown in FIG. 4A, the ECG sensing electrodes 112a, 112b, 112c, 112d are located over the rib cage, just under the breast area. For example, the ECG sensing electrodes 112 can be positioned circumferentially at the level of the xiphoid process. In implementations, the ECG sensing electrodes 112 are circumferentially placed along a transverse plane at the level of the xiphoid process. The left-side and right-side electrodes 112c, 112d are positioned on the midaxillary line. The anterior electrode 112a is positioned right of the sternum in the mid-clavicular line. The posterior electrode 112b is positioned between about 4 cm to about 12 cm left of the center of the spine, more particularly between about 6 cm to about 10 cm left of the center of the spine, and more particularly about 8 cm left of the center of the spine. Accordingly, two ECG channels produced using this arrangement can include a side-side (SS) ECG channel, and a front-back (FB) ECG channel, as shown in FIGS. 4B and 4C, for example.

In some examples, the four ECG sensing electrodes 112 used in this configuration are configured as two ECG leads that project onto a transverse plane 402 an angle that is substantially orthogonal. In examples, the four ECG sensing electrodes are configured as two ECG leads that project onto the transverse plane 402 (FIG. 4D) an angle of between 50 and 150 degrees. For example, wherein a first lead extends from a first geometrical center of a first one of the first pair of ECG sensing electrodes to a second geometrical center of a second pair of ECG sensing electrodes. The second lead extends from a third geometrical center of a first one of the second pair of ECG sensing electrodes to a fourth geometrical center of a second one of the second pair of ECG sensing electrodes. In this regard, projections of the first and second leads onto the transverse plane 402 of the patient 102 comprises a substantially orthogonal angle. In some examples, projections of the first and second leads onto a coronal plane 404 of the patient 102 comprises a substantially orthogonal angle.

In some examples, two or more ECG channels can be provided based on dynamically pairing at least two ECG sensing electrodes selected from the plurality of ECG sensing electrodes 112 disposed about the patient's thorax. For example, a first ECG sensing electrode 112a can be dynamically paired with a second ECG sensing electrode 112b to form a first ECG channel, and a third ECG sensing electrode 112c can be dynamically paired with a fourth ECG sensing electrode 112d to form a second ECG channel. In these implementations, the dynamic pairing can be based on a predetermined software ECG lead selection process where a best set of ECG channels can be automatically determined during live monitoring of the patient's ECG based on the patient's current activity status, body posture, time of day, and noise detected on the ECG channels, among other factors.

In further examples, three or more ECG channels can be provided based on dynamically pairing the at least four ECG sensing electrodes 112. For example, referring to FIG. 4A, a first side ECG sensing electrode 112c can be dynamically paired with an anterior ECG sensing electrode 112a to form a first ECG channel, a second side ECG sensing electrode 112d can be dynamically paired with a posterior ECG sensing electrode 112b to form a second ECG channel, the first side ECG sensing electrode 112c can be dynamically paired with the second side ECG sensing electrode 112d to form a third ECG channel, and the anterior ECG sensing electrode 112a can be dynamically paired with the posterior ECG sensing electrode 112b to form a fourth ECG channel. In these implementations, the dynamic pairing can be based on a predetermined ECG lead selection process where a best set of ECG channels can be automatically determined during live monitoring of the patient's ECG based on the patient's current activity status, body posture, time of day, and noise detected on the ECG channels, among other factors.

Referring again to FIG. 2, as discussed above, embodiments of the garment 110 may include a plurality of sensing electrode receptacles 212 that position the sensing electrodes 112 at predetermined anatomical positions in accord with various configurations as discussed above. In some examples, each of the sensing electrode receptacles 212 may be color coded to correspond to a matching color-coded sensing electrode 112, such that the sensing electrodes 112 can be correctly assembled into the sensing electrode receptacles 212 so as to arrange the sensing electrodes 112 at the predetermined anatomical positions to achieve the desired one or more ECG leads, as discussed above. In some examples, each sensing electrode 112 may be provided with a colored indicator (e.g., a marker or colored component) that matches the color associated with one of the sensing electrode receptacles 212. For example, each sensing electrode receptacle may include one or more components that have the matching color. Thus, the sensing electrode receptacles 212 and the sensing electrodes 112 may be arranged as color-coded pairs to facilitate easy, correct assembly of the sensing electrode system into the garment 110.

The sensing electrode receptacles 212 may be implemented in a variety of different ways, examples of which are discussed in more detail below. In certain examples, the sensing electrode receptacles can be configured to allow for non-destructive removable installation of the sensing electrodes 112, thereby allowing the sensing electrodes 112 to be removed from the garment 110 when necessary or desirable, for example, when laundering the garment 110. Furthermore, the sensing electrode receptacles can be configured to allow for easy, correct installation of the sensing electrodes 112.

Referring to FIGS. 5A-C, there is illustrated an example of a sensing electrode receptacle 500 that may be used to implement any of the sensing electrode receptacles 212. In this example, the casing of the sensing electrode receptacle 500 includes an annular holder 502 that is attached to an attachment device 504. In some examples, the annular holder 502 is configured as the securement device for the sensing electrode receptacle 500, as discussed further below. In many examples, the sensing electrodes 112 are at least approximately circular in shape. Accordingly, a benefit of providing the annular holder 502 includes providing a casing that corresponds to the shape and size of the sensing electrode 112, making it easier for the patient to correctly install the sensing electrode 112 in the sensing electrode receptacle 500. The attachment device 504 is configured to secure the holder 502 to the garment 110. The attachment device 504 may be secured to the fabric 510 in a variety of different ways, using permanent or removable attachment mechanisms. In the example shown in FIG. 5A, the attachment device 504 is adhesively secured to the fabric 510 using an adhesive 518. Additionally or alternatively, the attachment device 504 is stitched to the fabric portion 510 using industrial sewing thread such as a mildew-resistant, nylon twisted filament industrial sewing machine thread with a denier size of 235-255.

As shown in FIGS. 5A and 5C, in some examples, the holder 502 is an annular ring of predetermined shape and dimension. For example, an inner diameter of the annular ring is in a range of about 2 centimeters to about 8 centimeters. For example, an inner diameter of the annular ring is in a range of about 4.5 centimeters to about 5.5 centimeters. For example, the inner diameter of the annular ring is in a range of about 4.5 centimeters to about 6.5 centimeters. In an example implementation, the inner diameter of the annular ring is about 3 centimeters.

For example, the annular ring is about 0.1 centimeters to about 2 centimeters thick. For example, the annular ring is about 0.5 centimeters to about 1 centimeters thick. In an example implementation, the thickness of the annular ring is about 0.2 centimeters. The annular ring is configured to form an opening 506 into which a sensing electrode 112 is received. For example, the opening is shaped and dimensioned corresponding to the shape and dimension of the sensing electrode 112, as noted in further detail below. In some examples, the opening 506 has a diameter of in a range of about 3 centimeters to about 5 centimeters. For example, the opening 506 has a diameter in a range of about 3.5 centimeters to about 4 centimeters. For example, the opening 506 has a diameter in a range of about 3.7 centimeters to about 4.3 centimeters. In an example implementation, the opening 506 has a diameter of about 3 centimeters. The annular holder 502 may be positioned around a corresponding opening 508 in a fabric portion 510 of the garment 110. Thus, a benefit of the opening 506 in the annular holder 502 is that it allows for the sensing electrode 112 to be received in an aligned manner relative to the opening 508 in the fabric portion 510. Alternatively or additionally, such opening 506 allows for easy installation of the sensing electrode 112 to contact the patient's skin and minimize noise and/or rotational or shifting movement of the sensing electrode 112 when compared to existing or conventional features or techniques. The opening 506 thus avoids situations where a sensing electrode 112 is inadvertently positioned off-center relative to the sensing electrode receptacle 500. A patient who is assembling the sensing electrode 112 via such opening 506 will be provided with clear visual and tactile feedback as to whether the sensing electrode 112 is correctly placed at the predetermined anatomical location of the patient.

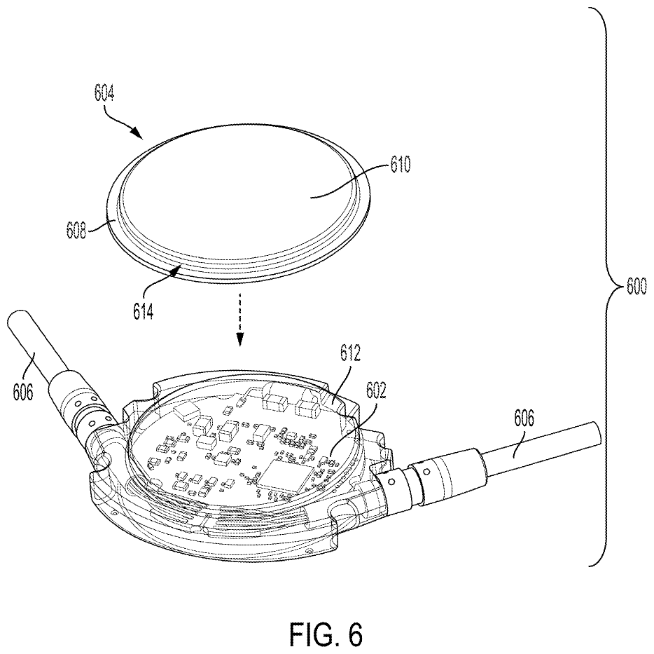

In an implementation, the sensing electrode 112 and sensing electrode receptacle 500 are configured so that when the sensing electrode 112 is inserted into the sensing electrode receptacle 500 via the opening 506 in the holder 502, at least a portion of the sensing electrode 112 (such as the skin-contacting electrode surface 610 discussed below with reference to FIG. 6, for example) will protrude above an edge of the receptacle 500, e.g., extend up to or through the opening 506 and/or up to or through the corresponding opening 508 in the garment 110 to contact the patient's skin. For example, the portion of the sensing electrode 112 that will protrude up to or through the opening 506 and up to or through the corresponding opening 508 in the garment 110 may be in a range of about 0.1 centimeters to about 0.2 centimeters. For example, the portion of the sensing electrode 112 that will protrude up to or through the opening 506 and up to or through the corresponding opening 508 in the garment 110 may be in a range of about 0.1 centimeters to about 0.5 centimeters. For example, the portion of the sensing electrode 112 that will protrude up to or through the opening 506 and up to or through the corresponding opening 508 in the garment 110 may be in a range of about 0.5 centimeters to about 1 centimeter. In some implementations, the sensing electrode 112 may be shaped and sized so as to be substantially flush with the edge of the receptacle 500. .

In examples, the opening 508 in the garment 110 corresponds to one of the predetermined anatomical locations on the patient's thorax (when the garment is worn by the patient). Thus, each sensing electrode receptacle 500 positions a respective sensing electrode 112 to contact the patient's skin at a corresponding predetermined anatomical location so as to detect ECG signals from the patient and, together with the other sensing electrode(s) 112 provide one or more ECG leads, as discussed above.

In some examples, the holder 502 includes one or more features that function as a securement device to removably secure the sensing electrode 112 at least partially within the sensing electrode receptacle 500 and as a guide to align the sensing electrode with one of the predetermined anatomical locations on the patient's thorax. As noted above, in an example, the sensing electrode 112 is inserted into the holder 502 via the opening 506. For example, the sensing electrode 112 may be inserted such that the skin-contacting surface 610 (see FIG. 6) protrudes on the opposing side of the garment (the side of the garment facing the patient). For example, the sensing electrode 112 may be inserted such that about 0.1 centimeters to about 0.5 centimeters of the thickness (depth) of sensing electrode 112 protrudes on the opposing side of the garment (the side of the garment facing the patient). For example, the sensing electrode 112 may be inserted such that about 0.1 centimeters to about 0.7 centimeters of the sensing electrode 112 protrudes on the opposing side of the garment (the side of the garment facing the patient). In some implementations, the sensing electrode 112 may be shaped and sized so as to be substantially flush with the edge of the receptacle 500.

In examples, the holder 502 is configured to secure the sensing electrode at least partially within the sensing electrode receptacle 500, as discussed above, while still allowing the sensing electrode to be easily and non-destructively removed from the sensing electrode receptacle 500 at any time. Thus, the sensing electrode 112 may be repeatedly installed into and removed from the sensing electrode receptacle 500. In some examples, the sensing electrode 112 can be press fit into the holder 502 to secure the sensing electrode 112 in the sensing electrode receptacle 500. For example, such press fit is when the holder 502 and the opening 506 is sized such that the sensing electrode 112 fits snugly into the sensing electrode receptacle 500 and is held in place by friction forces acting between the surface of the annular holder 502 in contact with the circumferential side or edge of the sensing electrode 112. The diameter of the opening 506 may be sized based on a known size of the sensing electrodes 112 to be used in the sensing electrode system. For example, the diameter of the opening may be in a range of about 4.3 centimeters to 6.2 centimeters); in some examples, about 4.5 centimeters, 5 centimeters, or 6 centimeters, within reasonable tolerances. The opening 508 in the fabric portion 510 of the garment 110 may be similarly sized or may have a smaller diameter than that of the opening 506. The opening 508 may be sufficiently sized to allow at least a portion of the sensing electrode 112 to contact the patient's skin to detect the ECG signals; whereas the opening 506 may need to be large enough to allow installation of the sensing electrode 112. In some examples, the opening 508 may have a diameter in a range of about 1.2-2 inches (3.048-5.8 centimeters), or in a range of about 1.4-1.8 inches (3.556-4.572 centimeters), or in a range of about 1.45-1.6 inches (3.683-4.064 centimeters), within reasonable tolerances. In the example illustrated in FIGS. 5A and 5C, the openings 506, 508 are circular. In some examples, the sensing electrodes 112 may be circular, and therefore, the openings 506, 508 may be circular. However, in other examples, the openings 506 and/or 508 may not be circular and may instead be oval, square, polygonal, rectangular, an irregular closed shape, or other geometric shape.

Referring to FIG. 5B, in an implementation, the annular holder 502 includes a pair of holder portions 512, 514 that are coupled together. For example, the holder 502 includes a first holder portion 512 that is secured to the fabric portion 510 of the garment 110 using the attachment device 504, as discussed above. In this example, the holder 502 further includes a second holder portion 514 that is positioned on an opposite side of the garment, such that the fabric 510 is sandwiched between the two holder portions 512, 514 as shown in FIG. 5B. In some examples, the second holder portion 514 is coupled to the first holder portion 512. For example, the holder 502 can include one or more fasteners 516, such as snaps, hooks, or another fastening mechanism, that attach the second holder portion 514 to the first holder portion 512. The attachment may be permanent or removable. Additionally or alternatively, the second holder portion 514 is secured to the fabric 510 of the garment 110 using a second attachment device that is the same as or similar to the attachment device 504.

In some examples, the first holder portion 512 includes the opening 506 that allows the sensing electrode 112 to be installed in the sensing electrode receptacle 500, and the second holder portion 514 includes a second, corresponding opening (not shown) that allows the sensing electrode to contact the patient's skin. A benefit of providing for the first holder portion 512 and second holder portion 514, as well as their corresponding openings 506 allows for easy installation of the sensing electrode 112 via the two openings so that the sensing electrode 112 contacts the patient's skin. An advantage of the two openings is that it helps secure the sensing electrode 112 to be immovable and thereby minimizes noise and/or rotational or shifting movement of the sensing electrode 112 when compared to existing or conventional features or techniques. Additionally or alternatively, such features helps avoid situations where a sensing electrode 112 is inadvertently positioned off center relative to the sensing electrode receptacle 500. A patient assembling the sensing electrode 112 via such features will be provided with clear visual and tactile feedback as to whether the sensing electrode 112 is correctly placed at the predetermined anatomical location of the patient.

In implementations, depending on certain design choices, the second opening in holder portion 514 can be the same size as opening 506 in holder portion 512, or smaller or larger than such opening 506. The first holder portion 512 may function as a securement device to removably secure the sensing electrode 112 in the sensing electrode receptacle 500. The second holder portion 514 may function as a guide to align the sensing electrode 112 with one of the predetermined anatomical locations on the patient's thorax when the garment 110 is worn. These features provide the benefits and advantages discussed above, such as making it easier for the patient to correctly install the sensing electrode 112 in the sensing electrode receptacle 500, preventing misalignment of the sensing electrode with respect to the sensing electrode assembly and/or the corresponding anatomical location on the patient's thorax, and providing the patient with clear visual and tactile feedback as to whether the sensing electrode 112 is correctly installed, for example. In some examples, the first and second holder portions 512, 514 are similarly sized, for example, having the same diameters and height, within reasonable tolerances. In other examples, the second holder portion 514 has a lower profile (lower height measured in a dimension extending orthogonally away from the surface of the fabric 510) than the first holder portion 512, as shown in FIG. 5B. The sensing electrode receptacle 500 may be arranged on the garment 110 such that the second holder portion 514 is on the side of the garment that contacts the patient's body (interior side). Such an arrangement provides a benefit in that the lower-profile structure may feel more comfortable against the patient's skin.

Referring again to FIG. 5A, in certain examples, the attachment device 504 is made of a flexible material, such as a fabric, Nylon, polymer, or flexible plastic, for example. The holder 502 may be made of a rigid or semi-rigid material. A semi-rigid material is one that holds its shape, but has some ability to deform, for example, to accommodate the sensing electrode 112 when installed and/or during the installation and/or removal process. As such, a holder 502 that is semi-rigid may be stiff and solid, but not inflexible. In some examples, the holder 502 is made of a thermoplastic or polymer material. In other examples, the holder 502 is made from another type of plastic material or metal. Examples of materials that can be used for the holder 502 include, but are not limited to: silicone, thermoplastic rubber, thermoplastic polyurethane, thermoplastic elastomer, polyvinyl chloride, Nylon, polyacetal, polycarbonate, polypropylene, or acrylonitrile butadiene styrene. In examples, the holder 502 is permanently secured to the attachment device 504, for example, by heat bonding, ultrasonic bonding, adhesive, and/or another attachment process/mechanism.

The attachment device 504 may be secured to the fabric 510 in a variety of different ways, using permanent or removable attachment mechanisms. In the example shown in FIG. 5B, the attachment device 504 is adhesively secured to the fabric 510 using an adhesive (e.g., similar to adhesive 518 of FIG. 5A). Additionally or alternatively, the attachment device 504 is stitched to the fabric 510 using industrial sewing thread such as a mildew-resistant, nylon twisted filament industrial sewing machine thread with a denier size of 235-255.

According to certain examples, each sensing electrode 112 includes an ECG sensing electrode assembly 600. Several components of an example of an ECG sensing electrode assembly 600, as may be used to implement any of the sensing electrodes 112 for use in a wearable medical device 100, are illustrated in an exploded view in FIG. 6. The ECG sensing electrode assembly 600 may be removably disposed at least partially within any of the sensing electrode receptacles 212, including the sensing electrode receptacle 500. As shown in FIG. 6, in some examples, the ECG sensing electrode assembly 600 includes a circuit board 602 that includes active ECG processing circuitry configured to digitize an ECG signal from a patient 102 wearing a wearable cardiac monitoring device/defibrillator, for example, in which the ECG sensing electrode assembly 600 is installed. The ECG sensing electrode assembly 600 further includes an ECG sensing electrode 604. The ECG sensing electrode 604 may comprise or consist of tantalum or any other suitable electrode material. The ECG sensing electrode 604 and the circuit board 602 are configured to be electrically coupled to one or more cables 606 (e.g., a wire or collection of wires) that are part of the cabling harness discussed above. In examples, the ECG sensing electrode 604 includes a peripheral region 608 and a surface 610 that is configured to contact the patient's skin to detect the ECG signals, as discussed above. In some examples, the surface 610 is raised relative to the peripheral region 608 to allow the surface 610 to extend through the openings 506, 508 to contact the patient's skin, as discussed above. In examples, the skin-contacting surface 610 of the sensing electrode 604 has a diameter in a range of about 3.5-4.5 centimeters, for example, about 3.8 centimeters or about 4 centimeters, within reasonable tolerances. The ECG sensing electrode assembly 600 further includes a housing 612 that encapsulates or partially encapsulates the circuit board 602. The peripheral region 608 of the ECG sensing electrode 604 can be coupled to the housing 612 or may be part of the housing 612. In examples, the ECG sensing electrode assembly 600 has dimensions (excluding the cables 606) in ranges of about 4-5 centimeters tall (e.g., about 4.5 centimeters tall) by about 4.5-5.5 centimeters wide (e.g., about 5 centimeters wide) by about 0.5-1.5 centimeter deep (e.g., about 1 centimeter deep). In examples, the ECG sensing electrode assembly 600 has dimensions (excluding the cables 606) in ranges of about 4-5.5 centimeters tall (e.g., about 4.7 centimeters tall) by about 5.5-6.5 centimeters wide (e.g., about 6 centimeters wide) by about 0.5-1.5 centimeter deep (e.g., about 1 centimeter deep).

In some examples, two cables 606 connect to the circuit board 602, one to carry signals to the medical device controller 120 of the wearable medical device 100 in which the ECG sensing electrode assembly 600 is installed, and another to receive signals from another ECG sensing electrode assembly 600 and pass these signals on to the medical device controller 120. If an ECG sensing electrode assembly 600 is last in a series of electrically connected ECG sensing electrode assemblies 600, the main circuit board 602 receives only a single cable 606 to carry signals back to the medical device controller 120.

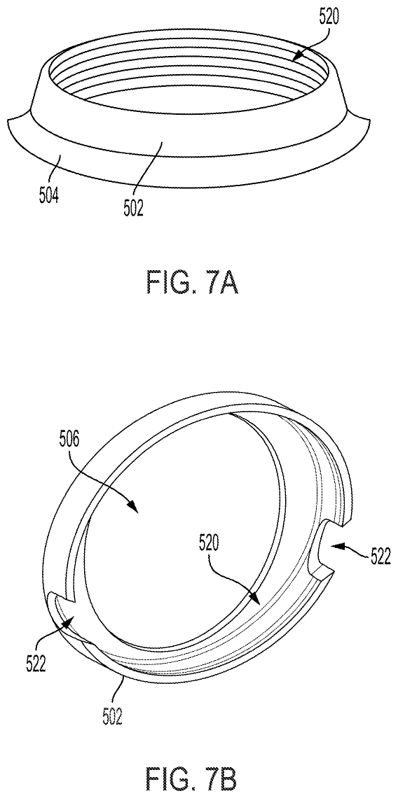

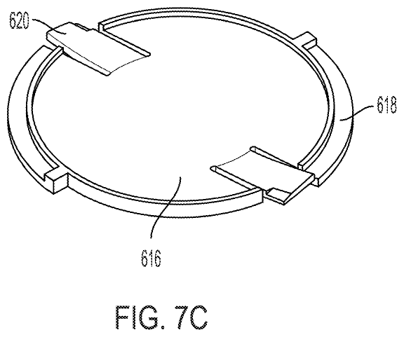

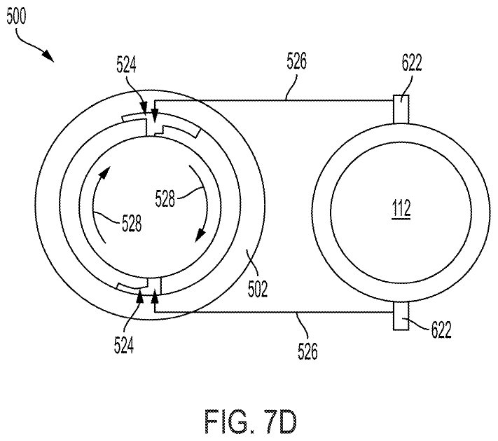

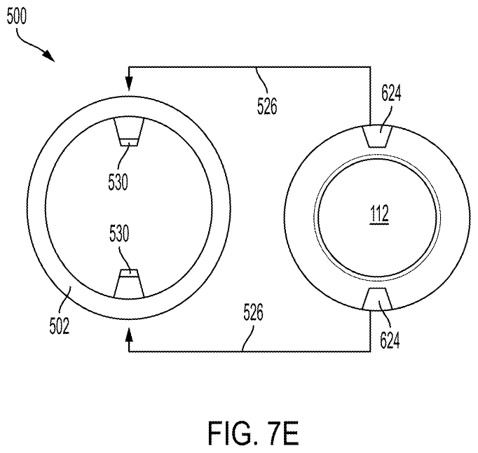

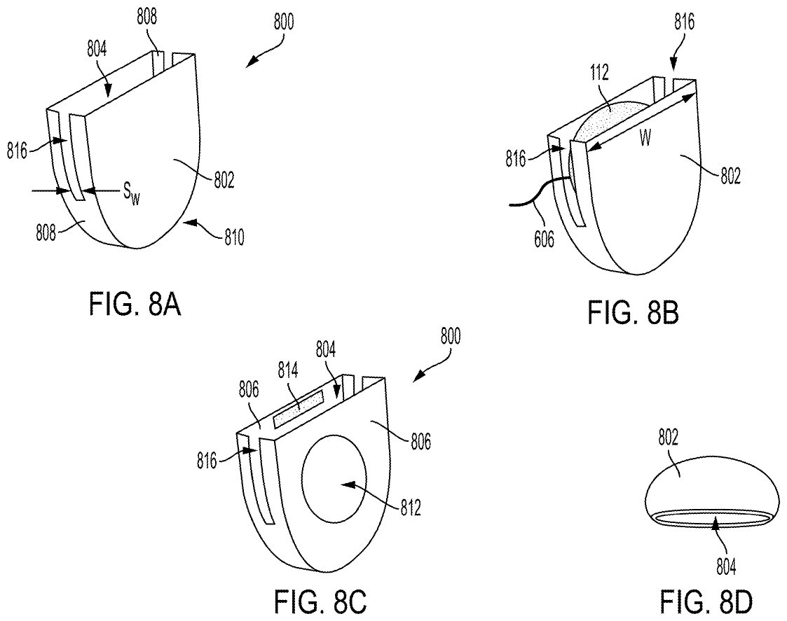

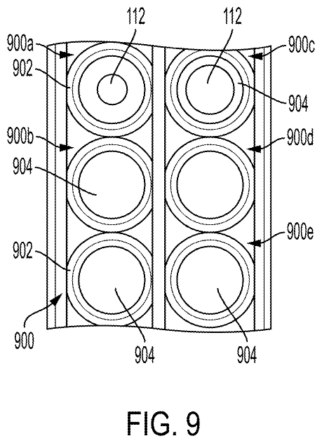

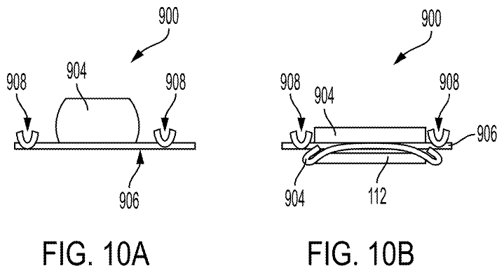

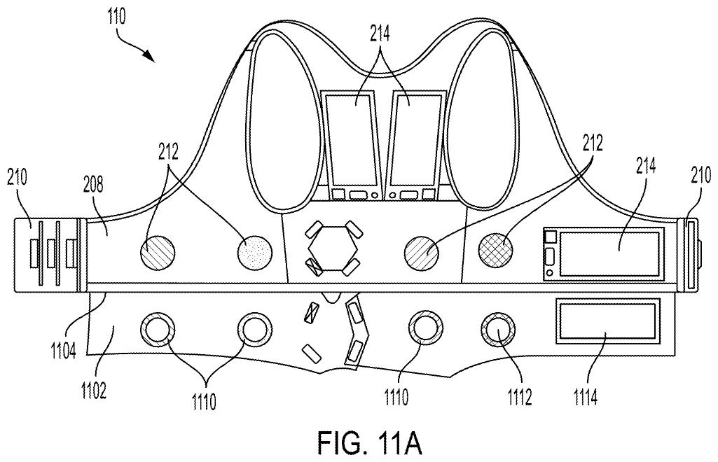

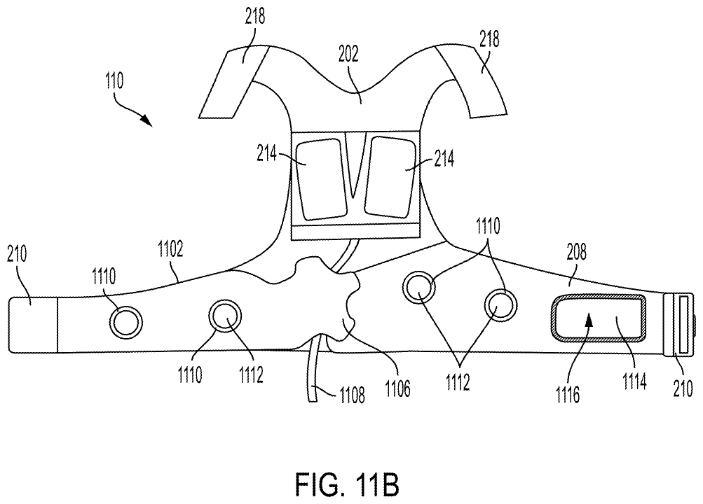

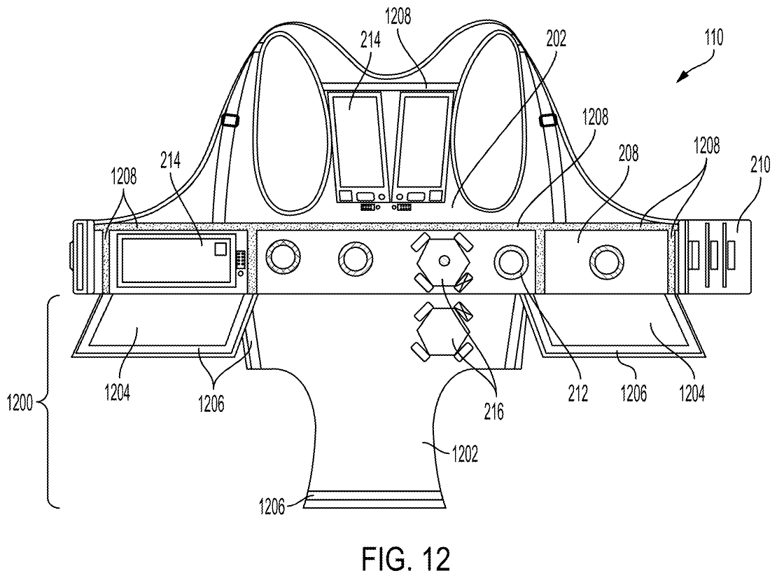

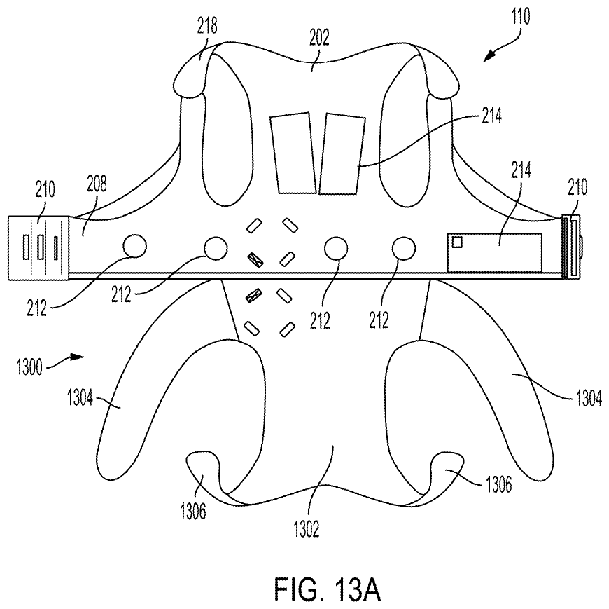

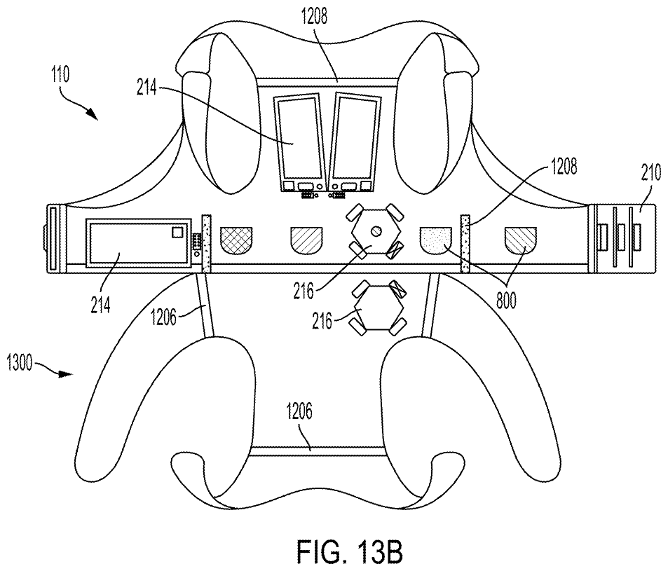

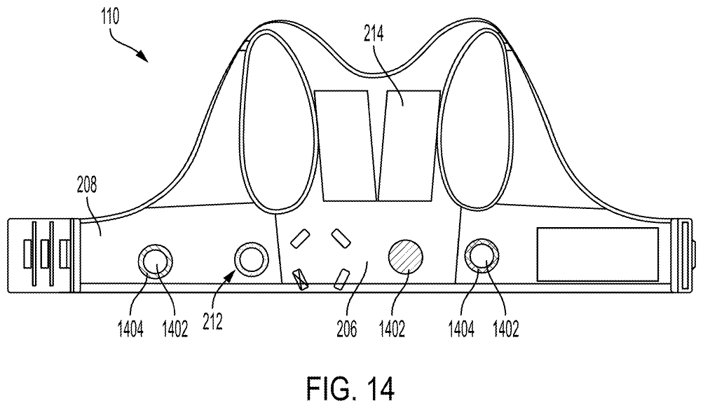

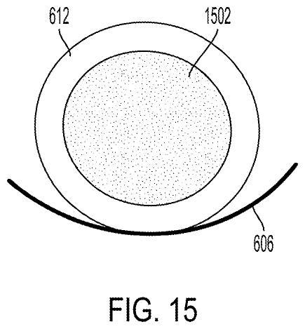

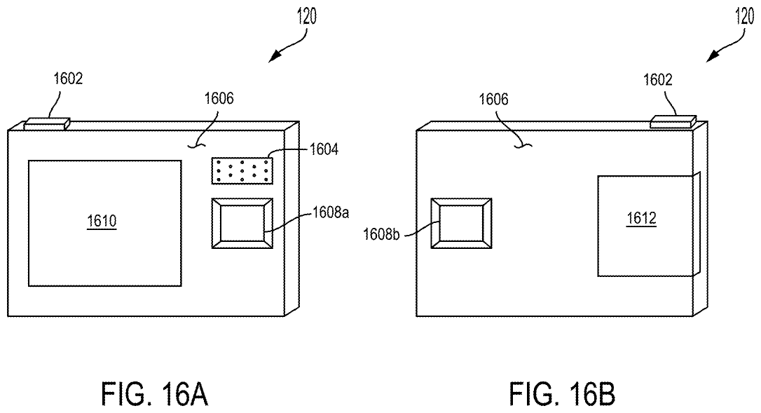

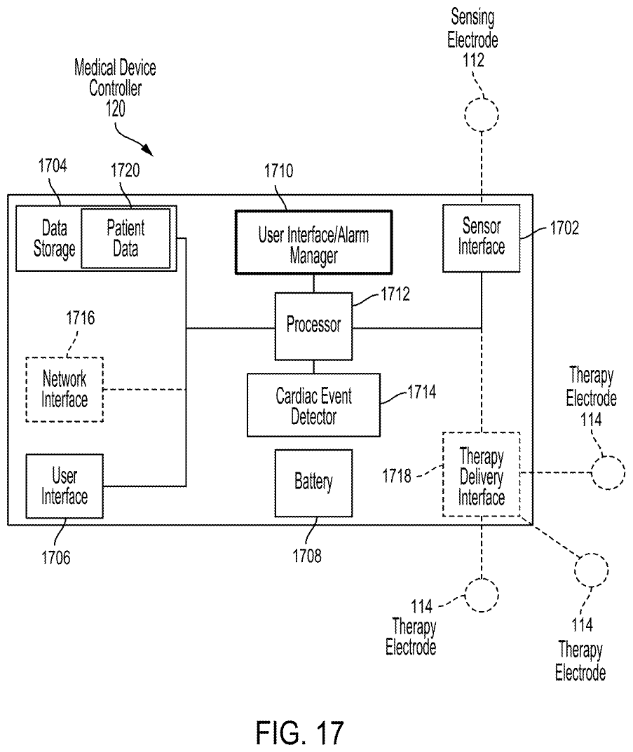

Referring to FIGS. 7A—according to certain examples, the sensing electrode receptacle 500 further includes a lock that is configured to inhibit movement of the sensing electrode (once installed) separate from the sensing electrode receptacle 500. Benefits of providing the lock include preventing rotational (e.g., “spinning”) and/or lateral (e.g., side-to-side) movement of the sensing electrode within the sensing electrode receptacle 500. The lock, when engaged, may also (or alternatively) prevent vertical movement (e.g., into and out of the sensing electrode receptacle) of the sensing electrode 112, such that the sensing electrode receptacle does not rely on friction forces, or friction forces alone, to secure the sensing electrode. Instead, the sensing electrode 112 is secured through engagement of the lock. A benefit of providing the securement device includes not having to rely on friction to secure the sensing electrode within the holder 502. Thus, the sizing of the annular holder 502, and the opening 506, may be more flexible and accommodate different sizes of sensing electrodes. In addition, for patients that may have reduced strength and/or dexterity, providing the securement device may make it easier to secure the sensing electrode without having to use as much force to install the sensing electrode as may be needed in examples where friction force is a primary mechanism by which the sensing electrode is held in place in the sensing electrode receptacle. A further benefit of the lock includes providing tactile and visual feedback to the patient that the sensing electrode 112 is properly secured within the electrode receptacle 500. In some examples, the lock includes one or more features formed in the holder 502 that are configured to engage mating features on the sensing electrode 112 to lock the sensing electrode in place in the sensing electrode receptacle 500, as discussed further below.

Referring to FIG. 7A, in some implementations, to provide the lock, the holder 502 includes threads, for example, one or more grooves and/or corresponding ridges (collective referred to as mating features 520) formed on an interior surface of the holder 502. The mating features 520 are configured to engage corresponding mating features 614 formed on the ECG sensing electrode assembly 600. For example, referring again to FIG. 6, the corresponding mating features 614 are formed as one or more groove(s) and/or corresponding ridge(s) on the periphery of the ECG sensing electrode 604. When the ECG sensing electrode assembly 600 is installed in the sensing electrode receptacle 500, the ECG sensing electrode assembly 600 can twist into place securely. For example, the ECG sensing electrode assembly 600 can be rotated such that the mating features 520 on the holder 502 engage the corresponding mating features 614 on the ECG sensing electrode assembly 600, and lock the ECG sensing electrode assembly 600 into the sensing electrode receptacle 500. In certain examples, a rotation of a quarter turn, a half turn, or one or more full turns is sufficient to engage the locking mechanism. When installed in the sensing electrode receptacle 500, the surface 610 of the ECG sensing electrode 604 extends up to or through the opening 508 in the garment 110 to contact the patient's skin, as discussed above.

In the example illustrated in FIG. 6, the cables 606 extend out from sides of the housing 612. Accordingly, in some examples, the holder 502 of the sensing electrode receptacle 500 includes features to accommodate and optionally secure the cables 606. For example, referring to FIG. 7B, in some examples, the holder 502 (e.g., holder portion 512) includes recesses 522 formed on its sides to allow the cables 606 to pass through. In some examples, the recesses 522 are sized and shaped to secure the cables 606. For example, the cables press snap into the recesses 522, such that they are held in place by friction. In examples, the recesses 522 have a width in a range of about 3 millimeters to 1.5 centimeters (e.g., to include a jacket around the cable connector to the sensing electrode 212), for example, about 50 millimeters, within reasonable tolerances. In some instances, the recesses 522 act both as cable guides (holding the cables 606 in place) and as part of the securement device and/or lock for the sensing electrode receptacle 500. For example, when the cables 606 are secured into the recesses 522, rotation of the ECG sensing electrode assembly 600 within the sensing electrode receptacle 500 is substantially prevented. In addition, by securing the cables 606, the ECG sensing electrode assembly 600 is also secured in the sensing electrode receptacle 500. Thus, in examples, the recesses 522 act (optionally in concert with other features of the sensing electrode receptacle 500) as a securement device to removably secure the sensing electrode 112. In other examples, as discussed above, the recesses 522 act (optionally in concert with other features of the sensing electrode receptacle 500) as a lock to lock the ECG sensing electrode assembly 600 in place, via engagement with the cables 606, once the ECG sensing electrode assembly 600 has been installed in the sensing electrode receptacle 500. Thus the recesses 522 prevent movement of the ECG sensing electrode assembly 600 separate from the sensing electrode receptacle 500.

In the example shown in FIG. 7B, the sensing electrode receptacle 500 includes side recesses 522 to accommodate the cables 606 extending from the sides of the ECG sensing electrode assembly 600, as discussed above. In other examples; however, the cables 606 extend from a surface of the ECG sensing electrode assembly 600 opposite to the surface 610 of the ECG sensing electrode 604. In such examples, the cables extend through the opening 506 in a direction away from the patient's body towards an external side of the garment 110. As discussed further below, in some examples, the garment 110 includes one or more features to secure the cables 606. Benefits of providing features to the secure the cables 606 include preventing the cables from dangling and potentially being caught or snagged on something that could potentially pull the cables loose from the sensing electrodes 112 and/or the connection pod 130, preventing the cables from getting tangled, and holding the cables away from the patient's skin, which may be more comfortable for the patient and reduce noise in the ECG signals.

Referring to FIG. 7C, in some examples, the ECG sensing electrode assembly 600 includes a base 616 that is part of or coupled to the housing 612 or peripheral region 608 of the ECG sensing electrode 604. The base 616 is removably rotatably securable to the holder 502 of the sensing electrode receptacle 500, as discussed above. In the illustrated example, the base 616 includes retention flanges 618 that, when the base 616 is rotated into place in the holder 502 of the sensing electrode receptacle 500, fit against an outer surface of the holder 502. Examples of the base 616 also include one or more locking tabs 620 which snap upward into tab openings formed in the holder 502 to prevent the base 616 (and therefore the ECG sensing electrode assembly 600) from being rotated and removed from the holder 502 unless the locking tabs 620 are depressed.

For example, referring to FIG. 7D, in some examples, the holder 502 includes tab openings 524 that are configured to receive and engage with locking tabs 622 on the (or locking tabs 620 on the base 616). The locking tabs 622 and tab openings 524 are configured to align the ECG sensing electrode 112 with the sensing electrode receptacle 500 when the ECG sensing electrode 112 is placed into the holder 502, as indicated by arrows 526. A predetermined amount of rotation (such as a ¼ turn as discussed above, for example), as indicated by arrows 528, can then cause the ECG sensing electrode 112 to click or snap securely into place, thus locking the ECG sensing electrode 112 into the sensing electrode receptacle 500.

Additionally or alternatively, tabs 530 can be provided on the holder 502 and configured to engage with corresponding tab openings 624 provided on the ECG sensing electrode 112 (e.g., on the base 616 or other portion of the ECG sensing electrode assembly 600). The tabs 530 can allow the ECG sensing electrode 112 to click or snap securely into place (e.g., the tabs 530 click or snap into the tab openings 624) once the ECG sensing electrode 112 is pushed into the holder 502. The tabs (530, 620, and/or 622) and corresponding tab openings (524 and/or 624) offer a benefit of providing tactile and visual feedback to the patient that the sensing electrode 112 is properly aligned and secured within the electrode receptacle 500.

FIGS. 8A-D illustrate another embodiment of a sensing electrode receptacle 800 that may be used for any of the sensing electrode receptacles 212. In the illustrated example, the casing of the sensing electrode receptacle 800 is configured as a pouch 802 having an opening 804 to receive a sensing electrode 112 (or ECG sensing electrode assembly 600). The sensing electrode 112 may be slid into and out of the pouch 802 via the opening 804. A benefit of providing the pouch 802 includes providing an easy-to-use receptacle for the sensing electrode that does not require substantial strength or dexterity for the patient to insert the sensing electrode 112. As noted above, the sensing electrode can simply be slid into the pouch 802. Another benefit includes providing a self-aligning feature-because the patient slides the sensing electrode into the pouch 802, the pouch prevents the sensing electrode from being off-center or misaligned with the sensing receptacle 800. Furthermore, the patient can easily see and feel that the sensing electrode is within the pouch 802, and thus the pouch 802 provides visual and tactile indicators to the patient that the sensing electrode 112 is correctly installed. In certain examples, the pouch 802 may be sized such that the sensing electrode 112 fits snugly at least partially within pouch 802 and is held in place by friction. Thus, in some examples, the pouch 802 itself acts as the securement device to secure the sensing electrode 112 in the pouch 802. For example, the pouch 802 has a lateral dimension, W, that is slightly larger than the diameter of the sensing electrode 112. For example, the lateral dimension, W, may be in a range of about 4.5-6.5 centimeters, or about 5-6 centimeters, within reasonable tolerances.

In some examples, the pouch 802 includes a securement device, such as a lip or protruding rim on an interior of the pouch (not shown in FIGS. 8A-D) that engages the sensing electrode 112 when the sensing electrode is pushed into the pouch 802 to secure the sensing electrode 112 within the pouch 802. In some examples, the sensing electrode receptacle 800 includes a securement component 814 (FIG. 8C) that allows the opening 804 in the pouch 802 to be at least partially closed so as to retain and secure the sensing electrode 112 within the pouch 802. This offers the benefit of providing an easy-to-use feature that ensures that the sensing electrode is secured within the sensing electrode receptacle and cannot fall out as the patient moves or handles the garment. Further benefits of such securement devices include allowing the patient to quickly drop the sensing electrode into the pouch 802 to hold it in place, and then engage the securement device to ensure that the sensing electrode does not fall out of the pouch, making it easier for the patient to assemble the sensing electrodes into the sensing electrode receptacles. As shown in FIG. 8C, in some implementations the securement component 814 is attached to one of the side surfaces 806 of the pouch 802. The securement component 814 may include one or more fasteners, such as snaps or hook and loop fasteners, for example. In such instances, the securement component 814 includes a mating element attached to the opposing side surface 806 of the pouch.

According to certain embodiments, the pouch 802 has a partially semi-circular shape, as shown in FIGS. 8A-C, to accommodate circular sensing electrodes 112; however, in other examples, the pouch 802 has another shape. In examples, the pouch 802 includes a pair of opposing side surfaces 806 and a pair of opposing edge surfaces 808. In some instances, the edge surfaces 808 taper, such that the pouch 802 is wider (in a dimension orthogonal to the lateral dimension, W) at one end (e.g., an upper end) proximate the opening 804 than at its opposing (e.g., bottom) end 810. In other examples, the pair of opposing side surfaces 806 are joined together such that the edge surfaces 808 are eliminated or are negligible in width, as shown in FIG. 8D, for example.

The pouch 802 is attached to the garment 110 at locations corresponding to the predetermined anatomical locations on the patient's thorax (when the garment is worn), as discussed above. In some examples, the sensing electrode receptacle 800 includes an attachment device to secure the pouch 802 to the garment 110. For example, the pouch 802 can be adhesively attached (i.e., the attachment device includes an adhesive) to the garment. For example, one of the outer side surfaces 806 is adhered to the garment 110. In another example, the pouch 802 is stitched to the garment 110, or thermally or ultrasonically bonded to the garment 110. In other examples, one or more fasteners, such as snaps or hook and loop fasteners, for example, are used to secure the pouch 802 to the garment 110. In some examples, the pouch 802 is made of a flexible plastic or polymer material. In one example, the pouch 802 is made of silicone. Other materials that can be used for the pouch 802 include, but are not limited to: thermoplastic rubber, thermoplastic polyurethane, thermoplastic elastomer, polyvinyl chloride, Nylon, polyacetal, polycarbonate, polypropylene, or acrylonitrile butadiene styrene.

As shown in FIG. 8C, in some examples, the pouch 802 includes an opening 812 in one of its side surfaces 806. The opening 812 is configured to allow a surface of the sensing electrode 112 (e.g., the surface 610 of the ECG sensing electrode assembly 600) to protrude through the opening 812 to contact the patient's skin. A benefit of the opening 812 in the pouch 802 is that it allows for the sensing electrode 112 to be received in an aligned manner relative to the sensing electrode receptacle 800 and the anatomical location on the patient's thorax. Alternatively or additionally, the opening 812 allows for easy installation of the sensing electrode 112 to contact the patient's skin and minimize noise and/or rotational or shifting movement of the sensing electrode 112 when compared to existing or conventional features or techniques. The opening 812 thus avoids situations where a sensing electrode 112 is inadvertently positioned off-center relative to the sensing electrode receptacle 800. A patient who is assembling the sensing electrode 112 via such opening 812 will be provided with clear visual and tactile feedback as to whether the sensing electrode 112 is correctly placed at the predetermined anatomical location of the patient. In some examples, the opening 812 is circular in shape, corresponding to a circular sensing electrode 112;

however, in other examples, the opening may have a different shape. In examples in which the opening 812 is circular, the opening has a diameter in a range of about 3.5-4.5 centimeters; in some examples, about 3.8 centimeters or 4 centimeters, within reasonable tolerances. As discussed above, in some examples of the ECG sensing electrode assembly 600, the electrode surface 610 is raised relative to the peripheral region 608 of the ECG sensing electrode 604. In examples, the opening 812 is sized and configured such that the raised electrode surface 610 (skin-contracting electrode surface) protrudes through the opening 812, for example, by about 0.1 to 0.2 centimeters, and the ECG sensing electrode 604 engages with the opening 812 (e.g., via friction or via a rim on the opening 812) to secure the sensing electrode 112 in the sensing electrode receptacle 800. Thus the opening 812 prevent lateral movement of the sensing electrode 112 separate from the sensing electrode receptacle 800. In such examples, the opening 812 advantageously acts as a securement device and as a guide to align the sensing electrode 112 with the predetermined anatomical location on the patient's thorax.

In some examples, the sensing electrode receptacle 800 includes one or more cable guides to support and optionally secure the cables 606 and provide the benefits discussed above. For example, as shown in FIGS. 8A-C, the pouch 802 includes a pair of slits 816 formed in the edge surfaces 808. The slits 816 allow the cables 606 to extend out from the sensing electrode 112 through the slits, as shown in FIG. 8B, for example. In some examples, the slits 816 are sized such that the cables 606 may be held snugly by friction when pushed into the slits 816. For example, slits 816 may be a few millimeters in width (S_{W}), for example, in a range of 4-6 millimeters, or approximately 5 millimeters (within reasonable tolerances).

As discussed above, because the cables 606 may be secured to the ECG sensing electrode assembly 600 at predetermined locations, by securing the cables 606 (e.g., in the slits 816), the ECG sensing electrode assembly 600 (or sensing electrode 112) is both aligned relative to the sensing electrode receptacle 800 and secured at least partially within the sensing electrode receptacle 800. Accordingly, the slits 816 act, optionally in concert with other features of the sensing electrode receptacle 800, as a guide to align the ECG sensing electrode assembly 600 and/or as a securement device to secure the ECG sensing electrode assembly 600 in the pouch 802.

FIGS. 9, 10A, and 10B illustrate examples of another embodiment of a sensing electrode receptacle in accordance with aspects of the present disclosure. FIG. 9 illustrates an array of sensing electrode receptacles 900. In some examples, the sensing electrode receptacles 900 are used with the garment 110 individually, rather than in an array format as shown in FIG. 9; however, FIG. 9 is intended to illustrate various features of the sensing electrode receptacles 900.

As shown in FIG. 9, in one embodiment, each sensing electrode receptacle 900 includes an outer retaining portion 902 and a movable portion 904. These components operate in concert to removably secure the sensing electrodes 112 to the garment 110 in a manner that is easy for the patient to use and offers benefits of allowing the patient to easily and quickly both correctly install the sensing electrode and receive visible confirmation that the sensing electrode is correctly installed. The movable portion 904 is coupled to the outer retaining portion 902 and moves at least partially within a space formed by the retaining portion 902. In some examples, the movable portion 904 is integrally formed with the retaining portion 902. In some examples, the retaining portion 902 and the movable portion 904 are made of a flexible polymer or plastic material, such as silicone, for example. Examples of materials that can be used for the holder 502 include, but are not limited to: silicone, thermoplastic rubber, thermoplastic polyurethane, thermoplastic elastomer, polyvinyl chloride, Nylon, polyacetal, polycarbonate, polypropylene, or acrylonitrile butadiene styrene. In the illustrated example, the retaining portion 902 is circular in shape and forms a circular opening in which the movable portion 904 is disposed. However, in other examples, the retaining portion 902 may have a different shape. Sensing electrodes 112 are secured to the movable portion 902. Accordingly, the opening formed by the retaining portion 902 is sufficiently sized to accommodate the sensing electrode 112. For example, the opening has a diameter in a range of about 4 centimeters to about 8 centimeters, or about 4.5 centimeters to about 6.5 centimeters, within reasonable tolerances.

The retaining portion 902 is secured to the garment 110 at a location that, when the garment is worn by the patient 102, corresponds to one of the predetermined anatomical locations on the patient's thorax, as discussed above.

In some examples, the movable portion 904 is movable between a first position and a second position. In FIG. 9, sensing electrode receptacles 900a and 900b are shown with the movable portion 904 in the first position and sensing electrode receptacles 900c, 900d, and 900e are shown with the movable portion 904 in the second position. When the movable portion 904 is in the second position, a surface of the sensing electrode 112 (e.g., surface 610) is pushed up to or through the opening formed by the retaining portion 902 into a position in which it can be in contact with the patient's skin. In some examples, the surface of the sensing electrode 112 is held in contact with the patient's skin by the movable portion 904 when the movable portion is in the second position.

Referring to FIGS. 10A and 10B, in some examples, the retaining portion 902 (not shown in FIGS. 10A and 10B) is attached to a fabric portion 906 of the garment 110 on a side of the garment that is away from the patient's body when the garment 110 is worn by the patient 102. In such examples, the garment may include an opening (as discussed above with reference to FIG. 5A. for example) and the retaining portion 902 may be positioned around the opening in the garment. As such, when the movable portion 904 is in the second position, at least part of the movable portion 904, and the skin-contacting surface of the sensing electrode 112, extend through the opening in the garment towards the patient's body. FIG. 10A illustrates an example showing the movable portion 904 in the first position, away from the patient's body. FIG. 10B shows another example in which the movable portion 904 is in the second position, with the sensing electrode 112 pushed through an opening in the garment fabric 906 towards a position in which it can be in contact with the patient's skin.

The sensing electrode 112 may be removably installed in the sensing electrode receptacle 900 using any of a variety of attachment and securement mechanisms. In some examples, the sensing electrode 112 is adhesively secured to the movable portion 904. In other examples, the sensing electrode 112 is removably attached to the movable portion 904 using a fastener, such as hook and loop fasteners, for example. In other examples, the movable portion 904 includes a retaining rim or other mechanical feature that is configured to engage the sensing electrode 112 and secure the sensing electrode 112 to the movable portion 904. A benefit of providing such a mechanical feature that engages the sensing electrode 112 includes providing a mechanism to secure the sensing electrode, and thus prevent the sensing electrode from falling out of the sensing electrode receptacle 900, without requiring the patient to use an adhesive or fastener. Further benefits of such a mechanical securement feature include aligning the sensing electrode with the sensing electrode receptacle, and providing visual and/or tactile feedback to the patient that the sensing electrode is correctly installed within the sensing electrode receptacle 900.

In some examples, the sensing electrode receptacle 900 further includes one or more cable guides 908 to hold the cables 606 that are attached to the sensing electrode 112, as discussed above. In the example illustrated in FIGS. 10A and 10B, the sensing electrode receptacle 900 includes a pair of cable guides 908, with one guide positioned on each side of the movable portion 904 to receive the cables 606 that extend outwardly from the sides of the ECG sensing electrode assembly 600 discussed above, for example. In the illustrated example, the cable guides 908 include C-shaped clips that are configured to hold the cables 606 when the cables are press fit into the clips. In some examples, the cable guides 908 are directly secured to the garment fabric 906; however, in other examples, the cable guides 908 are coupled to (or form part of) the retaining portion 902. Similar to examples discussed above, in certain instances, the cable guides 908 advantageously further function (optionally in concert with other features of the sensing electrode receptacle 900) as alignment guides and/or securement devices for the sensing electrodes 112, providing the associated benefits discussed above.

As discussed above with reference to FIG. 2, the sensing electrode receptacles 212 (optionally implemented as any of the examples of the sensing electrode receptacles 500, 800, or 900) are disposed a various positions on the garment 110. In some examples, the sensing electrode receptacles are co-located with openings in the garment 110, such that skin-contacting surfaces of the sensing electrodes extend through the garment openings to contact the patient's body at the appropriate locations, as discussed above. In some such examples, the sensing electrode receptacles 212 are attached to the garment 110 on the external, or non-patient facing, surface of the garment. In other examples, as in the case of examples of the sensing electrode receptacles 500, for example, the sensing electrode receptacles include portions on both sides of the garment 110. In particular, in some examples, the cable guides (e.g., cable guides 908) are positioned on the exterior side of the garment 110. Similarly, in examples of the sensing electrodes 500 and 800, where the cable guides may be part of the holder portion 512 or pouch 802, respectively, those components of the sensing electrode receptacles (e.g., the holder portion 512 or the pouch 802) are secured to the exterior side of the garment 110. These arrangements offer benefits including that the patient does not feel discomfort due to the cables 606 and/or larger components of the sensing receptacles 212 contacting their skin. In addition, to minimize noise in the ECG signals, it is preferable that the cables 606 are held away from the patient's body, to the extent possible. However, it is also preferable to minimize protrusions on the exterior surface of the garment 110 (e.g., due to the cables 606 and/or components of the sensing electrode receptacles 212) that could potentially snag on the patient's clothing worn over the garment or on external objects.

Accordingly, in certain examples, the garment 110 includes a belt flap or covering component that can be removably attached on either the interior or exterior (depending on the configuration of the garment 110) sides of the garment 110 and cover at least parts of the sensing electrode receptacles 212 and/or the cables 606, thus keeping the cables 606 and/or components of the sensing electrode receptacles secure within the garment 110.

Referring to FIGS. 11A and 11B, there is illustrated an example of the garment 110 including a belt flap 1102 configured to be removably attached to the lower portion 206 of the body region 202 and/or one or both of the pair of side portions 208. In the example illustrated in FIGS. 11A and 11B, the belt flap 1102 is configured to be attached to the interior (patient-facing) side of the garment 110; however, in the other examples, the belt flap 1102 is configured to be attached to an exterior side of the garment 110. When attached, the belt flap 1102 is disposed over the plurality of sensing electrode receptacles 212. FIG. 11A shows an example of the garment 110 with the belt flap 1102 in the open, or unattached configuration, and FIG. 11B shows an example of the garment 110 with the belt flap 1102 in the closed, or attached, configuration. As shown in FIG. 11A, in some examples, the belt flap 1102 is permanently attached to the garment 110 along one or more edges 1104, even when in the open/“unattached” configuration, and can be folded up and removably secured to the lower portion 206 of the body region 202 and/or to one or both side portions 208 along one or more other edges to be in the closed/“attached” configuration. Accordingly, the belt flap 1102 includes one or more fastening mechanisms, such as snaps, hook and loop fasteners, buttons, or zippers, for example, to removably secure the belt flap 1102 to the garment 110 in the closed configuration.

In some examples, the belt flap 1102 includes panels 1106 that overlap in an envelope style when the belt flap is in the closed configuration, so as to lock over the plurality of sensing electrode receptacles 212 and onto the garment 110. The belt flap 1102 mayalso cover the hold in place various cables of the cabling harness (e.g., cables 606 attached to the sensing electrodes 212) and/or one or more cables 1108 that may connect the therapy electrodes 114 (which may be housed in the pockets 214 as discussed above) to the connection pod 130 and/or medical device controller 120, for example. Thus, the belt flap 1102 offers benefits of securely holding components of the sensing electrode system in place without inhibiting movement of the patient.

In some examples, the belt flap 1102 include alignment components 1110 that are placed to fit over corresponding sensing electrode receptacles 212 on the garment 110 when the belt flap 1102 is closed. Each of the alignment components 1110 surrounds a belt flap opening 1112 in the belt flap 1102 that allows the skin-contacting surface of the sensing electrode 112 installed in each sensing electrode receptacle 212 to contact the patient's skin through the belt flap opening 1112. The alignment components 1110 allow for the sensing electrodes 112 to be received in an aligned manner relative to the belt flap openings1112. Alternatively or additionally, the alignment components 1110 allow for easy installation of the sensing electrode 112 to contact the patient's skin and minimize noise and/or rotational or shifting movement of the sensing electrode 112 when compared to existing or conventional features or techniques. The alignment components 1110 help to avoid situations where a sensing electrode 112 is inadvertently positioned off-center relative to the sensing electrode receptacle 212. The alignment components 1110 mayprovide a patient who is assembling the sensing electrode 112 with clear visual and tactile feedback as to whether the sensing electrode 112 is correctly placed at the predetermined anatomical location of the patient.

The belt flap openings 1112 maybe sized based on the sizes of the sensing electrodes 112. For example, the belt flap openings 1112 (and thus an inner diameter of each of the alignment components 1110) have a diameter in a range of For example, the diameter of the opening may be in a range of about 4.3 centimeters to 6.2 centimeters); in some examples, about 4.5 centimeters, 5 centimeters, or 6 centimeters, within reasonable tolerances. In the illustrated example, the alignment components 1110 are circular to accommodate circular sensing electrodes 112, or sensing electrodes 112 having circular skin-contacting surfaces (e.g., surface 610 discussed above); however, in other examples the alignment components 1110 mayhave other shapes. In some examples, the alignment components 1110 include molded rigid or semi-rigid rings that are configured to press fit over the exposed portion of the sensing electrode 112 (e.g., the surface 610), thus further locking the sensing electrode in place and facilitating reliable skin contact at the predetermined anatomical location on the patient's thorax when the garment 110 is worn. Thus, the alignment components 1110 function, optionally in concert with other components of the sensing electrode receptacles 212 (as discussed above), as securement devices and/or guides for the sensing electrodes 112 that secure the sensing electrodes 112 in place and/or ensure correct installation and alignment of the sensing electrodes 112 in the sensing electrode receptacles 212. In some examples, the alignment components 1110 maybe color coded to matching sensing electrodes 112 and sensing electrode receptacles 212. Examples of materials that can be used for the alignment components 1110 include, but are not limited to, thermoplastic rubber, thermoplastic polyurethane, thermoplastic elastomer, polyvinyl chloride, Nylon, polyacetal, polycarbonate, polypropylene, silicone, or acrylonitrile butadiene styrene.

As shown in FIG. 11B, in some examples, the belt flap 1102 mayfurther includes a therapy electrode alignment device 1114 configured to fit over the therapy electrode 114 housed in the pocket 214 that is located on the side portion 208 of the garment (as discussed above with reference to FIG. 2). The therapy electrode alignment device 1114 mayinclude a thin molded rim that surrounds an opening 1116 to facilitate ensuring that the therapy electrode pocket 214 is exposed and in full contact with the patients' skin when the belt flap 1102 is closed and the garment 110 is worn.

As discussed above, in the example illustrated in FIGS. 11A and 11B, the belt flap 1102 is configured to be attached on the interior of the garment 110. In other examples, in which the belt flap 1102 is configured to be attached to the exterior of the garment 110, the belt flap 1102 may not include the alignment components 1110 and the therapy electrode alignment device 1114 since there is no need to expose the sensing electrodes 112 and/or therapy electrodes 114 on the exterior of the garment 110.

In some examples, the belt flap 1102 is replaced with a covering component that covers more of the body region 202 of the garment than does the belt flap 1102. FIG. 12 illustrates an example of a cover 1200 that can be used with examples of the garment 110. In the illustrated example, the cover 1200 includes three panels, namely, a central panel 1202 and two side panels 1204. When the cover 1200 is in the closed position, the side panels 1204 are attached to and cover at least parts of the side portions 208 of the garment 110. Similarly, when the cover 1200 is in the closed position, the central panel 1202 is attached to and cover at least part of the body region 202 of the garment 110. As discussed above with respect to the belt flap 1102, in some examples, the cover 1200 is permanently attached to the garment 110 along one or more edges, even when in the open position, and the panels 1202, 1204 maybe folded up and removably attached to the garment 110 along one or more other edges when the cover 1200 is closed.

In some examples, the cover 1200 includes one or more fastening mechanisms to removably secure the panels 1202, 1204 to the garment 110 when the cover is closed. In the example illustrated in FIG. 12, the cover 1200 includes hook and loop fasteners 1206. Accordingly, in such examples, the garment 110 is provided with corresponding hook and loop fasteners 1208 to allow the cover 1200 to be removably attached to the garment 110. In other examples, the hook and loop fasteners 1206, 1208 are replaced with other fastening mechanisms, such as snaps, buttons, zippers, etc. It will be appreciated that an arrangement of hook and loop fasteners 1206, 1208 maybe similarly applied to the belt flap 1102 to removably secure the belt flap 1102 to the garment 110. The cover may advantageously provide the benefits discussed above with reference to the belt flap 1102.

In the example shown in FIG. 12, the cover 1200 includes three panels 1202, 1204, as discussed above. However, in other examples, the cover 1200 includes more or fewer panels. FIGS. 13A and 13B illustrate an example in which a cover 1300 is a single unitary piece. In this example, the cover 1300 includes a body region 1302 configured to be attached to and at least partially cover the body region 202 of the garment 110, and a pair of side or wing portions 1304 configured to be attached to and at least partially cover the side portions 208 of the garment 110. In the illustrated example, the cover 1300 further includes a pair of shoulder portions 1306 configured to be attached to and at least partially cover the shoulder portions 218 of the garment 110. It will be appreciated that various other configurations of the covers 1200, 1300, and/or the belt flap 1102 maybe implemented in accord with the principles disclosed herein.

In the examples shown in FIGS. 12 and 13, the covers 1200, 1300 are configured to be attached to the exterior of the garment 110. Referring to FIG. 13B, and as discussed above, external attachment of the cover 1300 mayprovide several benefits. For example, where the sensing electrode receptacles 800 are used (FIG. 13B), when the cover 1300 is closed, the cover presses against the sensing electrode receptacles 800, pushing or holding the pouches 802 closed and facilitating retaining the sensing electrodes 112 within the pouches 802 and/or keeping the sensing electrodes 112 pressed into the openings 812 (FIGS. 8B and 8C) to contact the patient's skin.

In other examples, the covers 1200 and/or 1300 are configured to be attached to the interior of the garment 110. In such examples, the covers 1200 and/or 1300 include alignment components 1110 corresponding to the sensing electrodes 112, as discussed above with reference to FIGS. 11A and 11B. In some such examples, the covers 1200 and/or 1300 further include one or more therapy electrode alignment devices 1114 positioned corresponding to one or more of the pockets 214, depending on the configuration of the cover 1200/1300 and how many of the pockets 214 are covered by the cover 1200/1300.

Referring again to FIG. 11A, and to FIGS. 14 and 15, in some examples, the sensing electrode receptacles 212 are implemented using hook and loop fasteners. In some such examples, each of the sensing electrode receptacles 212 includes a first hook and loop fastener component 1402, such as a hook pad or a loop pad. Each of the sensing electrodes 112 is provided with a corresponding second hook and loop fastener component, such as loop pad or hook pad, to allow the sensing electrodes 112 to be removably secured to the sensing electrode receptacles 212 using the hook and loop fasteners. For example, referring to FIGS. 6 and 15, where the sensing electrodes 112 are implemented using the ECG sensing electrode assembly 600, the second hook and loop fastener component 1502 (e.g., a hook or loop pad) is provided on an exterior surface of the housing 612 that is opposite the skin-contacting surface 610 of the ECG sensing electrode 604. The sensing electrode receptacles 212 each include a mating first hook and loop fastener component 1502 (e.g., a loop or hook pad). The hook pad includes hooks configured to engage complementary loop fasteners in the loop pad. Thus, the ECG sensing electrode assembly 600 can be removably attached to the sensing electrode receptacle 212, with the skin-contacting surface 610 facing away from the garment 110 and towards the patient's body when the garment is worn. The second hook and loop fastener component 1502 is adhesively coupled to the housing 612, for example.

In some examples, the sensing electrode receptacles 212 include a securement component configured to align the sensing electrodes 112 with respect to the plurality of second hook and loop fastener components. A benefit to providing such a securement component is to assist the patient in achieving the proper placement and alignment of the sensing electrodes quickly and easily. A further benefit of providing the securement component is that it allows for the sensing electrode 112 to be received in an aligned manner relative to the first hook and loop fastener component 1402 and prevent situations where the sensing electrode 112 is inadvertently positioned off-center relative to the first hook and loop fastener component. Alternatively or additionally, the securement component allows for easy installation of the sensing electrode 112 to contact the patient's skin and minimize noise and/or rotational or shifting movement of the sensing electrode 112 when compared to other features or techniques. Furthermore, a patient who is assembling the sensing electrode 112 via the securement component will be provided with clear visual and tactile feedback as to whether the sensing electrode 112 is correctly placed at the predetermined anatomical location of the patient. In some examples, this securement component may be implemented as the alignment components 1110 on the belt flap 1102. As discussed above, the alignment components 1110 can be arranged and configured to press fit over the sensing electrodes 112. Thus, where the sensing electrode receptacles 212 include hook and loop fasteners, the alignment components 1110 operate to ensure that the sensing electrodes 112 are correctly positioned on the sensing electrode receptacles 212. In other examples, the securement components can be implemented in a manner similar to (or the same as) the alignment components 1110 discussed above, but be provided on one or more attachment panels that are separate from the belt flap 1102. In further examples, as shown in FIG. 14, the securement components 1404 can be implemented on the garment 110. For example, the securement component 1404 includes a semi-rigid securement component (e.g., a plastic or polymer ring) positioned around a respective first hook and loop fastener component 1402 and configured to engage a corresponding sensing electrode 112 to align the sensing electrode with the hook and loop fastener component 1402.

Thus, aspects and embodiments provide various garment configurations and implementation techniques and approaches for removably attaching ECG sensing electrodes to a garment to provide a wearable cardiac monitoring and/or treatment device that is easy to assemble and comfortable for long-term, continuous wear by a patient.

Wearable medical devices 100 including any of the features disclosed herein can be capable of continuous use by the patient 102. In some implementations, the continuous use can be substantially or nearly continuous in nature. That is, the wearable medical device can be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless be considered continuous use. For example, the wearable medical device can be configured to be worn by a patient for as many as 24 hours a day. In some implementations, the patient can remove the wearable medical device for a short portion of the day (e.g., for half an hour to bathe). In such an example, nearly continuous can include 23.5 hours a day of wear with a half hour removal period.

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other healthcare provider (HCP) provides specific instruction to the patient to stop use of the wearable medical device.

Regardless of the extended period of wear, the use of the wearable medical device can include continuous or nearly continuous wear by the patient as described above. For example, the continuous use can include continuous wear or attachment of the wearable medical device to the patient, e.g., through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (e.g., ECG information, including arrhythmia information, cardio-vibrations, etc.) and/or non-cardiac information (e.g., blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

Referring again to FIGS. 1A and 1B, various examples of the medical device 100 include the ECG sensing electrode system, optionally one or more therapy electrodes 114, the medical device controller 120, and the connection pod 130, optionally along with other components, as discussed above. The ECG sensing electrode system includes the plurality of sensing electrodes 112, at least some of which can be removably attached to the garment using any of the sensing electrode receptacles 212 discussed above, and others of which may be permanently integrated with the garment 110 (e.g., stitched or woven into the garment). In certain implementations, the sensing electrodes 112 can be associated with additional components disposed within a housing of the sensing electrode 112 (e.g., within the housing 612), such as accelerometers, acoustic signal detecting devices, and other measuring devices for recording additional physiological, motion, or posture parameters. For example, such additional components can also be configured to detect other types of patient physiological parameters and acoustic signals, such as tissue fluid levels, heart vibrations, lung vibrations, respiration vibrations, patient movement, and the like.

As discussed above, the medical device controller 120 can be operatively coupled to the sensing electrode system and receive ECG signals from the sensing electrodes 112. In some examples, the medical device controller 120 can also be operatively coupled to the therapy electrodes 114. For example, the therapy electrodes 114 can be assembled into the garment 110 (e.g., being removably housed in the pockets 414 discussed above), or, in some implementations, the therapy electrodes 114 can be permanently integrated into the garment 110. In an example, the medical device controller is operably connected to the patient interface pod 140 to allow a patient to interact with the medical device 100. For example, the patient can use the patient interface pod 140 to respond to activity-related questions, prompts, and surveys. In other examples, the medical device 100 may not include the patient interface pod 140, and a user interface may be incorporated within the medical device controller 120 and/or an external device (not shown in FIG. 1B). Examples of the medical device controller 120 are discussed further below with reference to FIGS. 16A, 16B, and 17.

In some examples, the therapy electrodes 114 can also be configured to include sensors configured to detect ECG signals as well as other physiological signals of the patient. The connection pod 130 can, in some examples, include a signal processor configured to amplify, filter, and digitize these cardiac signals prior to transmitting the cardiac signals to the medical device controller 120. One or more of the therapy electrodes 114 can be configured to deliver one or more therapeutic defibrillating shocks to the body of the patient 102 when the medical device 100 determines that such treatment is warranted based on the signals detected by the ECG sensing electrodes 112 and processed by the medical device controller 120. Example therapy electrodes 114 can include metal electrodes such as stainless-steel electrodes that include one or more conductive gel deployment devices configured to deliver conductive gel to the metal electrode prior to delivery of a therapeutic shock.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 114 and associated circuitry can be optionally decoupled from (or coupled to) or switched out of (or switched in to) the medical device. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

In some examples, the wearable medical device 100 can be configured to monitor other non-ECG physiologic parameters of the patient in addition to cardiac related parameters. For example, the wearable medical device can be configured to monitor, for example, pulmonary-vibrations (e.g., using microphones and/or accelerometers), breath vibrations, sleep related parameters (e.g., snoring, sleep apnea), tissue fluids (e.g., using radio-frequency transmitters and sensors), among others. Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, e.g., capable of providing therapeutic pacing pulses to the patient. In some examples, the ambulatory medical devices can be configured to monitor for and/or measure ECG metrics including, for example, heart rate (such as average, median, mode, or other statistical measure of the heart rate, and/or maximum, minimum, resting, pre-exercise, and post-exercise heart rate values and/or ranges), heart rate variability metrics, premature ventricular contraction (PVC) burden or counts, atrial fibrillation burden metrics, pauses, heart rate turbulence, QRS height, QRS width, changes in a size or shape of morphology of the ECG information, cosine R-T, artificial pacing, QT interval, QT variability, T wave width, T wave alternans, T-wave variability, and ST segment changes.

Component configurations other than those shown in FIG. 1B are possible. For example, the sensing electrodes 112 can be configured to be attached at various positions about the body of the patient 102. In some implementations, the sensing electrodes 112 can be adhesively attached to the patient 102. In some implementations, the sensing electrodes 112 and at least one of the therapy electrodes 114 can be included on a single integrated patch and adhesively applied to the patient's body. Further, in some examples, the medical device 100 may include further components not shown in FIG. 1B, such as one or more non-ECG physiological sensors (e.g., accelerometers, vibrational sensors, RF-based sensors, and other measuring devices for recording additional non-ECG physiological parameters), for example.

FIGS. 16A and 16B illustrate an example medical device controller 120. For example, the controller 120 includes a connector receptacle 1602 for connecting the sensing and/or therapy electrode components to the controller 120. The controller 120 includes a speaker 1604 for providing audio prompts to the subject and/or a bystander. The controller 120 includes circuitry as further described below with reference to FIG. 17. The circuitry is housed within a mechanical housing structure 1606 to protect the circuitry and other internal components of the controller 120 from physical damage, particle ingress, and/or water ingress. The controller includes one or more response buttons 1608a, 1608b. A patient wearing the wearable medical device 100 can communicate with the controller 120 via the buttons 1608a, 1608b. For example, if the device detects a life-threatening arrhythmia condition in the subject, the controller 120 can direct the patient to press the one or more buttons 1608a, 1608b. In some examples, the controller 120 can include a display screen 1610. For example, the display screen 1610 can be a touch-sensitive panel screen responsive to subject input in the form of touch or physical force applied to the screen. For example, the display screen 1610 can display controls and/or prompts to the patient and is responsive to the patient's touch or application of physical force on the displayed controls. The controller 120 can be powered by a removable battery 1708 (see FIG. 17) that is housed within a battery chamber 1612.

FIG. 17 illustrates an example component-level view of a medical device controller 120 that is configured to control components of the medical devices described herein as well as process signals received from, for example, one or more sensing electrodes 112 as described herein. As shown in FIG. 17, the medical device controller 120 can include a sensor interface 1702, a data storage 1704, a user interface 1706, at least one battery 1708, a user interface/alarm manager 1710, at least one processor 1712, and a cardiac event detector 1714. The medical device controller 120 may optionally further include a network interface 1716 and a therapy delivery interface circuit 1718.

In examples in which the medical device controller 120 includes treatment functionality, the therapy delivery circuitry 1718 can be configured to provide one or more therapeutic shocks to a patient via at least two therapy electrodes 114. For example, the therapy delivery circuitry 1718 can include, or be operably connected to, circuitry components that are configured to generate and provide an electrical therapeutic shock. The circuitry components can include, for example, resistors, capacitors, relays and/or switches, electrical bridges such as an H-bridge (e.g., including a plurality of insulated gate bipolar transistors or IGBTs), voltage and/or current measuring components, and other similar circuitry components arranged and connected such that the circuitry components work in concert with the therapy delivery circuitry and under control of one or more processors (e.g., processor 1712) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). In some examples, the capacitors can include a single film or electrolytic capacitor as a series connected device including a bank of the same capacitors. These capacitors can be switched into a series connection during discharge for a defibrillation pulse. For example, a single capacitor of approximately 140 μF or larger, or four capacitors of approximately 650 μF can be used. The capacitors can have a 1600 V_{DC} or higher rating for a single capacitor, or a surge rating between approximately 350 to 500 V_{DC} for paralleled capacitors and can be charged in approximately 15 to 30 seconds from a battery pack.

For example, each defibrillation pulse can deliver between 60 to 180 joules of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuitry 1718 can be configured to perform the switching and pulse delivery operations, e.g., under control of the processor 1712. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

In certain examples, the therapy delivery circuitry 1718 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 1704 can include one or more of non-transitory computer-readable media, such as flash memory, solid state memory, magnetic memory, optical memory, cache memory, combinations thereof, and others. The data storage 1704 can be configured to store code and data used for operation of the medical device controller 120. In certain examples, the data storage can include executable instructions that, when executed, cause the processor 1712 to perform one or more operations. In some examples, the data storage 1704 can be configured to store information such as ECG data as received via, for example, the sensor interface 1702. The data storage 1704 may further store patient data 1720, which may include various information about the patient 102 associated with a particular medical device 100.

In some examples, the network interface 1716 can facilitate the communication of information between the medical device controller 120 and one or more other devices or entities over a communications network. For example, where the medical device controller 120 is included in an ambulatory medical device, the network interface 1716 can be configured to communicate with a remote computing device such as a remote server or other similar computing device. The network interface 1716 can include communications circuitry for transmitting data in accordance with a Bluetooth® wireless standard for exchanging such data over short distances to an intermediary device. For example, such an intermediary device can be configured as a base station, a “hotspot” device, a smartphone, a tablet, a portable computing device, and/or other devices in proximity of the wearable medical device including the medical device controller 120. The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a WI-FI communications link based on the IEEE 802.11 standard.

The medical device controller 120 can also include at least one rechargeable battery 1508 configured to provide power to one or more components integrated in the medical device controller 120. The rechargeable battery 1708 can include a rechargeable multi-cell battery pack. In one example implementation, the rechargeable battery 1708 can include three or more 2200 mAh lithium ion cells that provide electrical power to the other device components within the medical device controller 120. For example, the rechargeable battery 1708 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 120.

The sensor interface 1702 can include physiological signal circuitry that is coupled to one or more sensors configured to monitor one or more physiological parameters of the patient. As shown, the sensors can be coupled to the medical device controller 120 via a wired or wireless connection. The sensors can include one or more ECG sensing electrodes 112, and optionally other sensors (not shown).

In certain implementations, the cardiac event detector 1714 can be configured to monitor a patient's ECG signal for an occurrence of a cardiac event such as an arrhythmia or other similar cardiac event. The cardiac event detector can be configured to operate under control of the processor 1712 to execute one or more methods that process received ECG signals from, for example, the sensing electrodes 112 and determine the likelihood that a patient is experiencing a cardiac event. The cardiac event detector 1714 can be implemented using hardware or a combination of hardware and software. For instance, in some examples, cardiac event detector 1714 can be implemented as a software component that is stored within the data storage 1704 and executed by the processor 1712. In this example, the instructions included in the cardiac event detector 1714 can cause the processor 1712 to perform one or more methods for analyzing a received ECG signal to determine whether an adverse cardiac event is occurring. In other examples, the cardiac event detector 1714 can be an application-specific integrated circuit (ASIC) that is coupled to the processor 1712 and configured to monitor ECG signals for adverse cardiac event occurrences. Thus, examples of the cardiac event detector 1714 are not limited to a particular hardware or software implementation.

In certain examples, the user interface 1706 and/or the user interface/alarm manager 1710 can include one or more physical interface devices such as input devices, output devices, and combination input/output devices and a software stack configured to drive operation of the devices. These user interface elements can render visual, audio, and/or tactile content. Thus, the user interface 1706 and/or the user interface/alarm manager 1710 can receive input or provide output, thereby enabling a user to interact with the medical device controller 120. In certain implementations, the user interface/alarm manager 1710 can be configured to manage alarm profiles and notify one or more intended recipients of events specified within the alarm profiles as being of interest to the intended recipients. These intended recipients can include external entities such as users (patients, physicians, and monitoring personnel) as well as computer systems (monitoring systems or emergency response systems). Certain functionality of the user interface/alarm manager 1710 can be implemented using hardware or a combination of hardware and software. For instance, in some examples, certain functionality of the user interface/alarm manager 1710 can be implemented as a software component that is stored within the data storage 1704 and executed by the processor 1712. In this example, the instructions included in the user interface/alarm manager 1710 can cause the processor 1712 to configure alarm profiles and notify intended recipients using the alarm profiles.

In some implementations, the processor 1712 includes one or more processors (or one or more processor cores) that each are configured to perform a series of instructions that result in manipulated data and/or control the operation of the other components of the medical device controller 120. In some implementations, when executing a specific process (e.g., cardiac monitoring), the processor 1712 can be configured to make specific logic-based determinations based on input data received and be further configured to provide one or more outputs that can be used to control or otherwise inform subsequent processing to be carried out by the processor 1712 and/or other processors or circuitry with which processor 1712 is communicatively coupled. Thus, the processor 1712 reacts to specific input stimulus in a specific way and generates a corresponding output based on that input stimulus. In some example cases, the processor 1712 can proceed through a sequence of logical transitions in which various internal register states and/or other bit cell states internal or external to the processor 1712 can be set to logic high or logic low. As referred to herein, the processor 1712 can be configured to execute a function where software is stored in the data store 1704 coupled to the processor 1712, the software being configured to cause the processor 1712 to proceed through a sequence of various logic decisions that result in the function being executed. The various components that are described herein as being executable by the processor 1712 can be implemented in various forms of specialized hardware, software, or a combination thereof. For example, the processor 1712 can be a digital signal processor (DSP) such as a 24-bit DSP. The processor 1712 can be a multi-core processor, e.g., having two or more processing cores. The processor 1712 can be an Advanced RISC Machine (ARM) processor such as a 32-bit ARM processor or a 64-bit ARM processor. The processor 1712 can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the scope of the appended claims. For example, it is to be understood that the present disclosure contemplates that, to the extent possible, one or more features of any embodiment can be combined with one or more features of any other embodiment.

Other examples are within the scope of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

What is claimed is:

1. An ambulatory cardiac device for providing comfortable, long-term continuous cardiac monitoring and treatment for arrythmia conditions, the device comprising:
a plurality of sensing electrodes configured to detect electrocardiogram (ECG) signals of an ambulatory patient;
a garment configured to be worn about the patient's thorax;
a plurality of sensing electrode receptacles configured to
dispose, via the garment, the plurality of sensing electrodes at a plurality of predetermined anatomical locations on the patient's thorax, and
maintain, via the garment, contact between the plurality of sensing electrodes and the plurality of predetermined anatomical locations despite movement of the patient's thorax, wherein each sensing electrode receptacle forms an opening in the garment and comprises
a securement device configured to allow for removable installation of a respective sensing electrode,
a lock configured to inhibit movement of the respective sensing electrode separate from the sensing electrode receptacle, and
a guide configured to align the respective sensing electrode with one of the plurality of predetermined anatomical locations of the patient's thorax through the opening; and
a plurality of removable therapy electrodes configured to deliver a treatment to the patient in response to the ambulatory cardiac device detecting a cardiac arrhythmia condition indicated by the ECG signals.

2. The device of claim 1, wherein the securement device includes a first annular holder at least partially surrounding the opening.

3. The device of claim 2, wherein the lock includes a plurality of grooves formed in a surface of the first annular holder, wherein at least one groove of the plurality of grooves engages the respective sensing electrode to lock the respective sensing electrode into the securement device.

4. The device of claim 2, wherein the guide includes a second annular holder coupled to the first annular holder and at least partially surrounding the opening.

5. The device of claim 4, wherein the first and second annular holders are made of a thermoplastic material.

6. The device of claim 2, wherein each sensing electrode receptacle further includes an attachment device coupled to at least one of the guide and the securement device, the attachment device configured to secure the sensing electrode receptacle to the garment.

7. The device of claim 6, wherein the attachment device is made of fabric.

8. The device of claim 1, wherein the plurality of sensing electrodes and the plurality of sensing electrode receptacles are configured as color-coded pairs, each color-coded pair including one sensing electrode and one sensing electrode receptacle.

9. The device of claim 1, wherein the garment comprises:
a body region;
a pair of side portions extending laterally from either side of a lower portion of the body region, the side portions being attachable to each other to form a waist for the garment; and
a pair of shoulder portions, each shoulder portion of the pair of shoulder portions extending between an upper portion of the body region and a respective one of the side portions.

10. The device of claim 9, wherein the garment further comprises a belt flap configured to be removably attached to at least one of the lower portion of the body region or the pair of side portions; and
wherein, when attached to the at least one of the lower portion of the body region or the pair of side portions, the belt flap is disposed over the plurality of sensing electrode receptacles.

11. The device of claim 9, wherein the garment further comprises a covering component configured to be removably attached to the upper portion of the body region and at least one of the lower portion of the body region or the pair of side portions; and
wherein, when attached to the upper portion of the body region and at least one of the lower portion of the body region or the pair of side portions, the covering component is disposed over the plurality of sensing electrode receptacles.

12. The device of claim 1, further comprising:
a cabling harness coupled to the plurality of sensing electrodes, the cabling harness including at least one wire electrically connected to each respective sensing electrode.

13. The device of claim 12, wherein each sensing electrode receptacle includes a cable guide configured to secure the at least one wire electrically connected to the respective sensing electrode.

14. The device of claim 1, wherein the plurality of sensing electrodes is a plurality of first sensing electrodes, and wherein the device further comprises:
at least one second sensing electrode permanently integrated with the garment.

15. The device of claim 14, wherein the at least one second sensing electrode is stitched or woven into the garment.

16. The device of claim 1, wherein the garment comprises a plurality of pockets configured to removably house the plurality of removable therapy electrodes.

17. The device of claim 1, wherein the securement device comprises a pouch secured to the garment, the pouch having a first opening to receive the respective sensing electrode; and
wherein the guide comprises a second opening in the pouch, the second opening being aligned with the opening in the garment and configured to permit the respective sensing electrode to contact the patient's skin at the one of the predetermined anatomical locations of the patient's thorax through the second opening.

18. The device of claim 17, wherein the pouch is made of a flexible polymer.

19. The device of claim 17, wherein the securement device further comprises a fastener configured to at least partially close the first opening to secure the respective sensing electrode within the pouch.

20. The device of claim 17, wherein the lock includes at least one protrusion formed on an interior of the pouch at an end of the pouch opposing the first opening, the at least one protrusion configured to engage the respective sensing electrode to secure the respective sensing electrode within the pouch.
